SEQUENCE LISTING (52 sequences)

SEQ ID NO: 1 - DNA, 18 bp, Synthetic construct
  source 1..18: mol_type = other DNA
gtcatcctgcacatttta

SEQ ID NO: 2 - DNA, 18 bp, Synthetic construct
  source 1..18: mol_type = other DNA
ttcatcctcgcctcactc

SEQ ID NO: 3 - DNA, 18 bp, Synthetic construct
  source 1..18: mol_type = other DNA
tccatgagtttccacagg

SEQ ID NO: 4 - DNA, 18 bp, Synthetic construct
  source 1..18: mol_type = other DNA
catcttgtcatcctgcac

SEQ ID NO: 5 - DNA, 18 bp, Synthetic construct
  source 1..18: mol_type = other DNA
cttgtcatcctgcacatt

SEQ ID NO: 6 - DNA, 18 bp, Synthetic construct
  source 1..18: mol_type = other DNA
ttgtcatcctgcacattt

SEQ ID NO: 7 - DNA, 18 bp, Synthetic construct
  source 1..18: mol_type = other DNA
tgtcatcctgcacatttt

SEQ ID NO: 8 - DNA, 18 bp, Synthetic construct
  source 1..18: mol_type = other DNA
tcatcctgcacattttaa

SEQ ID NO: 9 - DNA, 18 bp, Synthetic construct
  source 1..18: mol_type = other DNA
catcctgcacattttaat

SEQ ID NO: 10 - DNA, 18 bp, Synthetic construct
  source 1..18: mol_type = other DNA
atcctgcacattttaatt

SEQ ID NO: 11 - DNA, 18 bp, Synthetic construct
  source 1..18: mol_type = other DNA
tcctgcacattttaatta

SEQ ID NO: 12 - DNA, 18 bp, Synthetic construct
  source 1..18: mol_type = other DNA
cctgcacattttaattac

SEQ ID NO: 13 - skipped

SEQ ID NO: 14 - protein, 4 aa, Synthetic construct
SAQA

SEQ ID NO: 15 - protein, 11 aa, Synthetic construct
SDGTLAVPFKA

SEQ ID NO: 16 - protein, 743 aa, Synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 17 - protein, 7 aa, Synthetic construct
NRGTEWD

SEQ ID NO: 18 - protein, 7 aa, Synthetic construct
QAVRTSL

SEQ ID NO: 19 - protein, 7 aa, Synthetic construct
TLAVPFK

SEQ ID NO: 20 - protein, 7 aa, Synthetic construct
DSPAHPS

SEQ ID NO: 21 - DNA, 131 bp, Homo sapiens
  source 1..131: mol_type = genomic DNA
ctaaataaagatggctttttagtattaaaagtggaagaaaattacaggtaattatctttgacggtaaaaacgctgtaatcagcgggctacatgaaaaattactctaattatggctgcatttaagagaatgg

SEQ ID NO: 22 - DNA, 393 bp, Synthetic construct
  source 1..393: mol_type = other DNA
ctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatgg

SEQ ID NO: 23 - DNA, 257 bp, Synthetic construct
  source 1..257: mol_type = other DNA
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgca

SEQ ID NO: 24 - DNA, 21 bp, Synthetic construct
  source 1..21: mol_type = other DNA
aaagagaccggttcactgtga

SEQ ID NO: 25 - DNA, 23 bp, Synthetic construct
  source 1..23: mol_type = other DNA
gaaacccagcagacaatgtagct

SEQ ID NO: 26 - DNA, 52 bp, Synthetic construct
  source 1..52: mol_type = other DNA
aaagagaccggttcactgtgagcggccttgaaacccagcagacaatgtagct

SEQ ID NO: 27 - DNA, 57 bp, Synthetic construct
  source 1..57: mol_type = other DNA
tcggaaaagagaccggttcactgtgagcggccttgaaacccagcagacaatgtagct

SEQ ID NO: 28 - DNA, 57 bp, Synthetic construct
  source 1..57: mol_type = other DNA
aaagagaccggttcactgtgagcggccttgaaacccagcagacaatgtagctcagta

SEQ ID NO: 29 - DNA, 62 bp, Synthetic construct
  source 1..62: mol_type = other DNA
tcggaaaagagaccggttcactgtgagcggccttgaaacccagcagacaatgtagctcagta

SEQ ID NO: 30 - DNA, 18 bp, Synthetic construct
  source 1..18: mol_type = other DNA
agttggagcaagattatc

SEQ ID NO: 31 - DNA, 42 bp, Synthetic construct
  source 1..42: mol_type = other DNA
aatttttggagcaggttttctgacttcggtcggaaaacccct

SEQ ID NO: 32 - DNA, 11 bp, Synthetic construct
  source 1..11: mol_type = other DNA
aatttttggag

SEQ ID NO: 33 - DNA, 168 bp, Synthetic construct
  source 1..168: mol_type = other DNA
cccaatttcactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctgg

SEQ ID NO: 34 - DNA, 221 bp, Synthetic construct
  source 1..221: mol_type = other DNA
caggttttctgacttcggtcggaaaacccctcccaatttcactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctggcacgtgcggaccgagcggccgc

SEQ ID NO: 35 - DNA, 987 bp, Synthetic construct
  source 1..987: mol_type = other DNA
gcggccgcacgcgtggtaccctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggagctctaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcatcggaaaagagaccggttcactgtgagcggccttgaaacccagcagacaatgtagctcagtaagttggagcaagattatcaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctggcacgtgcggaccgagcggccgc

SEQ ID NO: 36 - DNA, 925 bp, Synthetic construct
  source 1..925: mol_type = other DNA
gcggccgcacgcgtggtaccctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggagctctaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcagtcatcctgcacattttaaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctggcacgtgcggaccgagcggccgc

SEQ ID NO: 37 - DNA, 987 bp, Synthetic construct
  source 1..987: mol_type = other DNA
gcggccgcacgcgtggtaccctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggagctctaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcatcggaaaagagaccggttcactgtgagcggccttgaaacccagcagacaatgtagctcagtagtcatcctgcacattttaaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctggcacgtgcggaccgagcggccgc

SEQ ID NO: 38 - DNA, 130 bp, Synthetic construct
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 39 - DNA, 141 bp, Synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 40 - DNA, 146 bp, Synthetic construct
  source 1..146: mol_type = other DNA
catgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 41 - DNA, 149 bp, Synthetic construct
  source 1..149: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctg

SEQ ID NO: 42 - protein, 736 aa, Synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 43 - DNA, 450 bp, Synthetic construct
  source 1..450: mol_type = other DNA
gcggccttaaagagaccggttcactgtgacagtaaaagagaccggttcactgtgagaatgaaagagaccggttcactgtgatcggaaaagagaccggttcactgtgagcggccttaaagagaccggttcactgtgacagtaaaagagaccggttcactgtgagaatgaaagagaccggttcactgtgatcggaaaagagaccggttcactgtgagcggccttgaaacccagcagacaatgtagctcagtagaaacccagcagacaatgtagctgaatggaaacccagcagacaatgtagcttcggagaaacccagcagacaatgtagctgcggccttgaaacccagcagacaatgtagctcagtagaaacccagcagacaatgtagctgaatggaaacccagcagacaatgtagcttcggagaaacccagcagacaatgtagctgcggcc

SEQ ID NO: 44 - DNA, 214 bp, Synthetic construct
  source 1..214: mol_type = other DNA
aaagagaccggttcactgtgacagtaaaagagaccggttcactgtgagaatgaaagagaccggttcactgtgatcggaaaagagaccggttcactgtgagcggccttgaaacccagcagacaatgtagctcagtagaaacccagcagacaatgtagctgaatggaaacccagcagacaatgtagcttcggagaaacccagcagacaatgtagct

SEQ ID NO: 45 - DNA, 42 bp, Synthetic construct
  source 1..42: mol_type = other DNA
aaagagaccggttcactgtgacttcggtcgcagtgaacccct

SEQ ID NO: 46 - DNA, 31 bp, Synthetic construct
  source 1..31: mol_type = other DNA
caggttttctgacttcggtcggaaaacccct

SEQ ID NO: 47 - DNA, 540 bp, Synthetic construct
  source 1..540: mol_type = other DNA
tgcatttaagagaatggagctctaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaagttggagcaagattatcaatttttggagaaagagaccggttcactgtgacttcggtcgcagtgaacccctcccaatttcactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctggcacgtgcggaccgagcggccgc

SEQ ID NO: 48 - DNA, 953 bp, Synthetic construct
  source 1..953: mol_type = other DNA
gagctctaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaactagtgcggccttaaagagaccggttcactgtgacagtaaaagagaccggttcactgtgagaatgaaagagaccggttcactgtgatcggaaaagagaccggttcactgtgagcggccttaaagagaccggttcactgtgacagtaaaagagaccggttcactgtgagaatgaaagagaccggttcactgtgatcggaaaagagaccggttcactgtgagcggccttgaaacccagcagacaatgtagctcagtagaaacccagcagacaatgtagctgaatggaaacccagcagacaatgtagcttcggagaaacccagcagacaatgtagctgcggccttgaaacccagcagacaatgtagctcagtagaaacccagcagacaatgtagctgaatggaaacccagcagacaatgtagcttcggagaaacccagcagacaatgtagctgcggccagttggagcaagattatcaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctggcacgtg

SEQ ID NO: 49 - DNA, 953 bp, Synthetic construct
  source 1..953: mol_type = other DNA
gagctctaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaagttggagcaagattatcaatttttggagcaggttttctgacttcggtcggaaaacccctactagtgcggccttaaagagaccggttcactgtgacagtaaaagagaccggttcactgtgagaatgaaagagaccggttcactgtgatcggaaaagagaccggttcactgtgagcggccttaaagagaccggttcactgtgacagtaaaagagaccggttcactgtgagaatgaaagagaccggttcactgtgatcggaaaagagaccggttcactgtgagcggccttgaaacccagcagacaatgtagctcagtagaaacccagcagacaatgtagctgaatggaaacccagcagacaatgtagcttcggagaaacccagcagacaatgtagctgcggccttgaaacccagcagacaatgtagctcagtagaaacccagcagacaatgtagctgaatggaaacccagcagacaatgtagcttcggagaaacccagcagacaatgtagctgcggcccccaatttcactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctggcacgtg

SEQ ID NO: 50 - DNA, 953 bp, Synthetic construct
  source 1..953: mol_type = other DNA
gagctctaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaagttggagcaagattatcaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctggactagtgcggccttaaagagaccggttcactgtgacagtaaaagagaccggttcactgtgagaatgaaagagaccggttcactgtgatcggaaaagagaccggttcactgtgagcggccttaaagagaccggttcactgtgacagtaaaagagaccggttcactgtgagaatgaaagagaccggttcactgtgatcggaaaagagaccggttcactgtgagcggccttgaaacccagcagacaatgtagctcagtagaaacccagcagacaatgtagctgaatggaaacccagcagacaatgtagcttcggagaaacccagcagacaatgtagctgcggccttgaaacccagcagacaatgtagctcagtagaaacccagcagacaatgtagctgaatggaaacccagcagacaatgtagcttcggagaaacccagcagacaatgtagctgcggcccacgtg

SEQ ID NO: 51 - DNA, 401 bp, Synthetic construct
  source 1..401: mol_type = other DNA
tgcatttaagagaatggagctcaaccgaataaggaactgtgctttgtgattcacatatcagtggaggggtgtggaaatggcaccttgatctcaccctcatcgaaagtggagttgatgtccttccctggctcgctacagacgcacttccgcaagttggagcaagattatcaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctggcacgtgcggaccgagcggccgc

SEQ ID NO: 52 - DNA, 31 bp, Synthetic construct; identical to SEQ ID NO: 46
  source 1..31: mol_type = other DNA

(19) United States
(12) Patent Application Publication
(10) Pub. No.: US 2026/0347858 A1
(43) Pub. Date: Nov. 5, 2026

(54) ANTISENSE OLIGONUCLEOTIDES AND ARTIFICIAL EXPRESSION CONSTRUCTS FOR EXPRESSING RNA IN INHIBITORY NEURONS

(71) Applicant: Allen Institute, Seattle, WA (US)

(72) Inventors: Bryan Gore, Redmond, WA (US); Edward Sebastian Lein, Mercer Island, WA (US); Boaz P. Levi, Seattle, WA (US); Jiatai Liu, Seattle, WA (US); Refugio Martinez, Seattle, WA (US); John K. Mich, Seattle, WA (US); Jonathan Ting, Lake Forest Park, WA (US)

(73) Assignee: Allen Institute, Seattle, WA (US)

(21) Appl. No.: 19/486,482

(22) PCT Filed: Jul. 18, 2024
(86) PCT No.: PCT/US2024/038566

§ 371 Date: Nov. 21, 2025

Related U.S. Application Data
(60) Provisional application No. 63/514,322, filed on Jul. 18, 2023.

(51) Int. Cl. C12N 15/113; A61K 9/00; A61P 25/08; C07K 14/005; C12N 5/0793; C12N 15/86

(52) U.S. Cl. CPC C12N 15/1138; A61K 9/0075; A61P 25/08; C07K 14/005; C12N 5/0619; C12N 15/86; C12N 2310/11; C12N 2310/315; C12N 2310/321; C12N 2510/00; C12N 2750/14122; C12N 2750/14143; C12N 2750/14145

(57) ABSTRACT

Antisense oligonucleotides and artificial expression constructs for modulating RNA expression in targeted central nervous system cell types are described. The artificial expression constructs can be used to express RNA in GABAergic neurons. Antisense oligonucleotides and artificial expression constructs can be used to rescue sodium voltage-gated channel function to treat disorders such as epilepsy, and more particularly, Dravet Syndrome, among many other uses.

### CROSS-REFERENCE TO RELATED APPLICATION

This application is a U.S. National Phase Patent Application based on International Patent Application No. PCT/US2024/038566, filed on Jul. 18, 2024, which claims priority to and benefit of U.S. Provisional Patent Application No. 63/514,322 filed Jul. 18, 2023, each of which is incorporated herein by reference in its entirety as if fully set forth herein.

### REFERENCE TO SEQUENCE LISTING

The Sequence Listing associated with this application is provided in XML format in lieu of a paper copy and is hereby incorporated by reference into the specification. The name of the file containing the Sequence Listing is 3IX3303.XML. The file is 57,344 bytes, was created on or about Nov. 17, 2025, and is being submitted electronically via Patent Center.

### FIELD OF THE DISCLOSURE

The current disclosure provides sequences encoding antisense oligonucleotides (ASO) and artificial expression constructs for selectively expressing RNA in inhibitory neurons. The ASO and artificial expression constructs can be used in the treatment of sodium voltage-gated channel alpha subunit 1 (SCN1A)-related disorders as well as for research purposes.

### BACKGROUND OF THE DISCLOSURE

To fully understand the biology of the brain, different cell types need to be distinguished and defined and, to further study them, artificial expression constructs that can label and perturb them need to be identified.

Further, there are numerous neurological disorders for which treatments are urgently needed. One class of such disorders arise from mutations in SCN1A, the gene that encodes the alpha subunit of voltage-gated sodium channel, Nav1.1. For example, an SCN1A mutation can cause disorders such as Dravet syndrome, simple febrile seizures, and intractable childhood epilepsy with generalized tonic-clonic seizures.

Epilepsy is a neurological disorder that occurs when the brain presents an enduring predisposition to generate two or more epileptic seizures. An epileptic seizure is a temporary disruption of brain function due to abnormal excessive or synchronous neuronal activity. Its manifestation may include periods of unusual behavior, sensations and sometimes loss of consciousness.

Dravet Syndrome (DS) is a rare and catastrophic form of intractable epilepsy that begins in infancy. Initially, the patient experiences prolonged seizures. In their second year, additional types of seizures begin to occur which coincide with a developmental decline. This leads to poor development of language and motor skills.

Children with DS are likely to experience multiple seizures per day. Epileptic seizures are more likely to result in death in patients having DS; 10 to 16% of patients diagnosed with DS die in childhood, particularly between two and four years of age. Additionally, patients are at risk of numerous associated conditions including orthopedic developmental issues, impaired growth, sleep and circadian rhythm impairments, and chronic infections.

Of particular concern, children with DS are susceptible to episodes of status epilepticus. Status epilepticus is a condition in which a seizure lasts for more than 5 minutes or multiple seizures occur close together within a 5 minute-period without recovery of consciousness between them. This severe condition is categorized as a medical emergency requiring immediate medical intervention, typically involving hospitalization. Frequent hospitalizations of children with DS can be distressing, not only to the patient but to family and care givers. The cost of care for DS is high as the affected children require constant supervision or institutionalization. Prolonged convulsive status epilepticus lasting more than 30 minutes can lead to substantial brain damage or it can be fatal.

At present, although a number of anticonvulsant therapies can be employed to reduce the instance of seizures in patients with DS, the results obtained with such therapies are typically poor and those therapies only produce partial cessation of seizures in most patients. Many of these anticonvulsants such as clobazam and clonazepam have undesirable side effects, which are particularly acute in pediatric patients. Furthermore, certain anticonvulsants (particularly sodium-channel blockers) can exacerbate seizures when administered at an incorrect dose or within an incorrect patient population.

Cell-type or cell-class specific gene delivery using non-pathogenic viral delivery is showing increasing promise for the treatment of diverse diseases. Inclusion of particular genetic elements, such as specific promoters, enhancers, or microRNA binding sites within the delivered vector, has been beneficial to provide specificity for gene expression within particular targeted cell types. For example, Dimidschstein and colleagues (Nat Neurosci 19 (12): 1743-1749, 2016) developed a viral delivery gene construct based on the adeno-associated virus (AAV) that resulted in selective expression of a gene within gamma-aminobutyric acid (GABA) ergic interneurons within the telencephalon, a cell type important in the treatment of epilepsy.

Expressing the SCN1A gene as a selective gene-delivery system can be restricted because of the packaging limit of the vector; for example, the large 6 kb SCN1A gene exceeds the packaging capacity of AAV (4.7 kb DNA). Thus, there remains an urgent and unmet need to provide corrected SCN1A gene function.

### SUMMARY OF THE DISCLOSURE

The current disclosure provides sequences encoding antisense oligonucleotides (ASO) that result in increased expression of functional sodium channel protein type 1 subunit alpha (SCN1A) s from the SCN1A gene. These ASO can be used in the treatment of SCN1A-related disorders, such as Dravet syndrome, simple febrile seizures, and intractable childhood epilepsy with generalized tonic-clonic seizures among others described elsewhere herein. In particular embodiments, increased expression of functional protein from the SCN1A gene within a cell rescues the sodium voltage-gated channel function of the cell. In particular embodiments, the ASO is encoded by the sequence as set forth in any one of SEQ ID NOs: 1-12 or 30 or by a sequence with at least 90%, 95%, 97%, 98%, or 99% sequence identity to any one of SEQ ID NOs: 1-12 or 30. In particular embodiments, the ASO is encoded by the sequence as set forth in SEQ ID NO: 1 or SEQ ID NO: 30.

The current disclosure also provides artificial expression constructs that selectively drive expression of RNA sequences (e.g., ASO) in inhibitory neurons. Particular embodiments of the artificial expression constructs utilize a U7 promoter and encode an ASO. In particular embodiments, the artificial expression construct also includes or encodes an enhancer and/or an miR binding site. In particular embodiments, these artificial expression constructs further encode an Sm binding domain and a 3′ box. In particular embodiments, the miR binding site includes an miR-128 binding site and/or an miR-221 binding site.

Particular embodiments include or encode: an enhancer, a U7 promoter, an miR binding site, an ASO, an Sm binding domain, and a 3′ box. In particular embodiments, the enhancer includes a DLX2.0 (3xhl56icore) enhancer or a hl56i core enhancer. In particular embodiments, the miR binding site includes an miR-128 binding site and/or an miR-221 binding site.

Particular embodiments provide or encode from 5′ to 3′: a DLX2.0 enhancer, a U7 promoter, an miR-128 binding site, an miR-221 binding site, an ASO, a consensus Sm sequence (smOPT), and a 3′ box.

Artificial expression constructs disclosed herein can be used as a targeted therapeutic for patients with SCN1A-related disorders in order to restore sodium voltage-gated channel function. In particular embodiments, the artificial expression constructs are utilized to selectively express ASO within inhibitory neurons.

### BRIEF DESCRIPTION OF THE FIGURES

Some of the drawings submitted herein may be better understood in color. Applicant considers the color versions of the drawings as part of the original submission and reserves the right to present color images of the drawings in later proceedings.

FIG. 1. Depiction of an exemplary construct including the DLX2.0 enhancer, a U7 promoter, microRNA (miR) binding sites, an encoded antisense, a consensus Sm sequence (smOPT), and 3′ box.

FIG. 2. Screening of U7-Scn1a-antisense via RNAscope and quantification. Mice at postnatal day P2 received bilateral intracerebroventricular injections of U7 variants. Four weeks post-injections, brains were harvested and processed for RNAscope detection for 4′, 6-diamidino-2-phenylindole (DAPI), inhibitory neuron marker Gad1, excitatory neuron marker Slc17a7, and U7-Scn1a-antisense. The layer 5 motor cortex was quantified for raw smOPT U7 signal intensity. CN4706 shows a high level of specificity and completeness in forebrain inhibitory neurons of the mouse motor cortex.

FIG. 3. High magnification of CN4706. CN4706 drives high specificity and completeness in inhibitory neurons in the cortex, hippocampus, and thalamus. RNAscope detection of DAPI, inhibitory neuron marker Gad1, excitatory neuron marker Slc17a7, and U7-Scn1a-antisense.

FIG. 4. Scn1a-antisense oligonucleotide (ASO) 1-3 are novel sequences targeting regions of Scn1a mRNA that show 100% conversation between human and mouse. Mice were injected with bilateral intracerebroventricular injections at postnatal day P2 with 20 ug of the indicated antisense oligonucleotide (2′-O-methoxyethyl (2′MOE) base+phosphorothioate backbone). ASO1, ASO2, and ASO3 did not show an increase in Scn1a mRNA expression relative to phosphate buffered saline (PBS)-injected control animals. In contrast, Scn1a-ASO1 (SEQ ID NO: 1) showed increased expression of Nav1.1 protein (the protein encoded by the SCN1A gene).

FIG. 5. Table of sequences encoding ASO disclosed herein.

FIG. 6. Sequences supporting the disclosure including 1xhl56i (core) (core of human 156i enhancer) (131 bp in length) (SEQ ID NO: 21), DLX2. 0 (3xhl56i core) (393 bp in length) (SEQ ID NO: 22), U7 promoter (SEQ ID NO: 23), miR-128 binding site coding sequence (SEQ ID NO: 24), miR-221 binding site coding sequence (SEQ ID NO: 25), Linked miR-128 & miR-221 binding site (1x2C) coding sequences (SEQ ID NOs: 26-29), U7 smOPT coding sequence (SEQ ID NO: 31), smOPT coding sequence (SEQ ID NO: 32), U7snRNAOPT with miRNA-218 binding site coding sequence (SEQ ID NO: 45), U7 snRNAOPT coding sequence (SEQ ID NO: 46), Mouse U7 3′ box coding sequence (SEQ ID NO: 33) 3′box coding sequence (SEQ ID NO: 34), CN4706 (987 bp between ITRs) (SEQ ID NO: 35), CN4992 (925 bp between ITRs) (SEQ ID NO: 36), CN4993 (987 bp between ITRs) (SEQ ID NO: 37), CN4705 (540 bp between ITRs) (SEQ ID NO: 47), CN4707 (953 bp between ITRs) (SEQ ID NO: 48), CN4708 (953 bp between ITRs) (SEQ ID NO: 49), CN4709 (953 bp between ITRs) (SEQ ID NO: 50), CN4769 (401 bp between ITRs) (SEQ ID NO: 51), Exemplary Plasmid Backbone 1—Left ITR (SEQ ID NO: 38), Exemplary Plasmid Backbone 1—Right ITR (SEQ ID NO: 39), Exemplary Plasmid Backbone 2—Left ITR (SEQ ID NO: 40), Exemplary Plasmid Backbone 2—Right ITR (SEQ ID NO: 41), PHP.eB capsid (SEQ ID NO: 16), AAV9 VP1 capsid protein (SEQ ID NO: 42), 8x2C (450 bp in length) coding sequence (SEQ ID NO: 43), and 4x2C (214 bp in length) coding sequence (SEQ ID NO: 44).

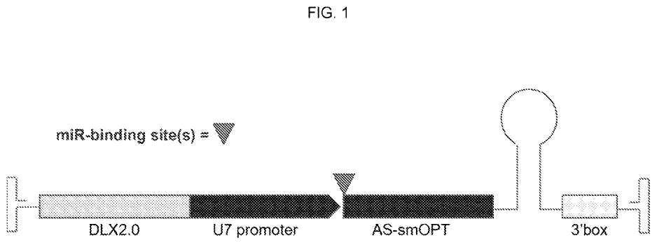

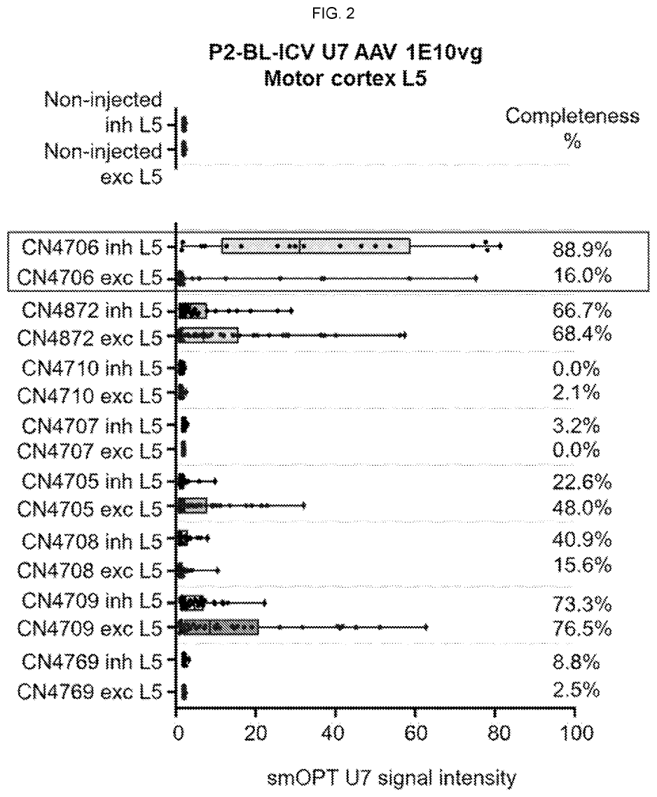

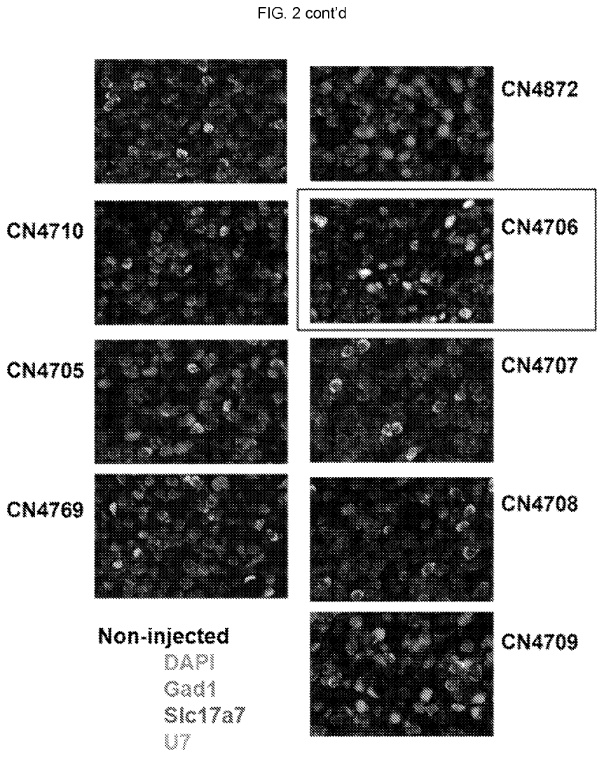

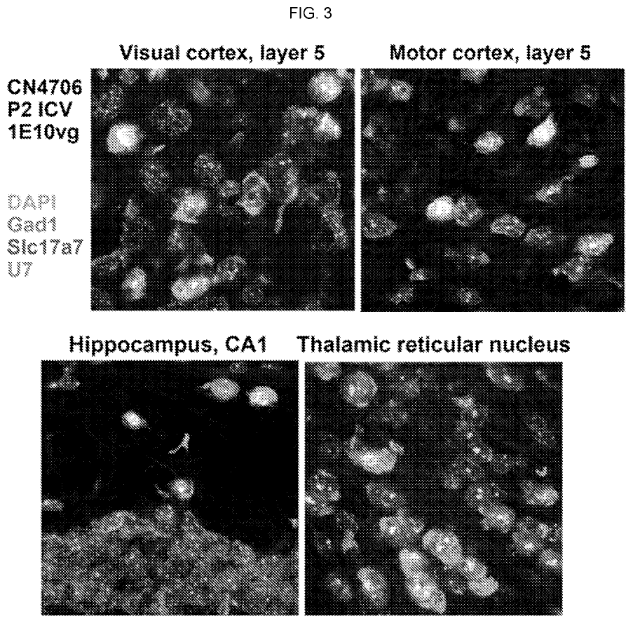

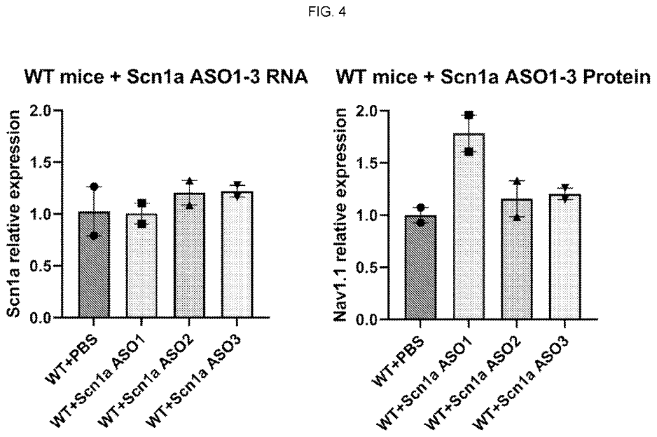

### DETAILED DESCRIPTION

The current disclosure provides sequences encoding antisense oligonucleotides (ASO) that increase expression of functional protein from the sodium channel protein type 1 subunit alpha (SCN1A) gene. These ASO can be used in the treatment of SCN1A-related disorders, such as Dravet syndrome, simple febrile seizures, and intractable childhood epilepsy with generalized tonic-clonic seizures among others described elsewhere herein. In particular embodiments, increased expression of functional protein from the SCN1A gene within a cell rescues the sodium voltage-gated channel function of the cell. In particular embodiments, the ASO is encoded by a sequence as set forth in any one of SEQ ID NOs: 1-12 or 30 or a sequence with at least 90%, 95%, 97%, 98%, or 99% sequence identity to any one of SEQ ID NOs: 1-12 or 30.

The current disclosure also provides artificial expression constructs that selectively drive expression of RNA in inhibitory neurons. Particular embodiments of the artificial expression constructs utilize a U7 promoter and encode an ASO. In particular embodiments, the artificial expression construct also includes or encodes an enhancer and/or a microRNA (miR) binding site. In particular embodiments, these artificial expression construct further encode an Sm binding domain and a 3′ box. In particular embodiments, the miR binding site includes an miR-128 binding site and/or an miR-221 binding site.

Particular embodiments provide or encode: an enhancer, a U7 promoter, an miR binding site, an ASO, an Sm binding domain, and a 3′ box. In particular embodiments, the enhancer includes a DLX2.0 (3xhl56icore) enhancer or a hl56i core enhancer. In particular embodiments, the miR binding site includes an miR-128 binding site and/or an miR-221 binding site.

In particular embodiments, the artificial expression construct includes or encodes (i) an enhancer, (ii) a U7 promoter, (iii) an miRNA binding site, (iv) an ASO, (v) an Sm binding domain, and (vi) a 3′ box.

In particular embodiments, the enhancer includes 1xhl56i (core) or DLX2.0 (3xhl56i (core)). In particular embodiments, the enhancer core (hl56icore) includes the sequence as set forth in SEQ ID NO: 21. In particular embodiments, this core is concatenated and can include 2, 3, 4, 5, 6, 7, 8, 9, or 10 copies of the core. In particular embodiments, a three-copy concatemer of the hl56i (core) includes the sequence as set forth in SEQ ID NO: 22, also referred to as DLX2.0.

In particular embodiments, the miR binding site includes an miR-128 binding site and an miR-221 binding site.

In particular embodiments, the ASO provides a beneficial effect in inhibitory neurons. In particular embodiments, the ASO is encoded by one or more of the sequences selected from SEQ ID NOs: 1, 2, 3, 4, 5, 6, 7, 8, 9, 10, 11, 12, and 30.

In particular embodiments, the Sm binding domain includes a consensus Sm sequence (SmOPT). In particular embodiments, the SmOPT is encoded by the sequence as set forth in SEQ ID NO: 31 or SEQ ID NO: 32.

In particular embodiments, the artificial expression construct includes or encodes (i) an enhancer (e.g., SEQ ID NOs: 21 or 22), (ii) a U7 promoter (e.g., SEQ ID NO: 23), (iii) a microRNA (miRNA) binding site, (iv) an ASO, (v) an Sm binding domain, and (vi) a 3′ box. In particular embodiments the miRNA binding site is encoded by the sequence as set forth in SEQ ID NOs: 24, 25, 26, 27, 28, 29, 43, and/or 44. In particular embodiments the ASO is encoded by the sequence as set forth in SEQ ID NOs: 1, 2, 3, 4, 5, 6, 7, 8, 9, 10, 11, 12, or 30. In particular embodiments the Sm binding domain is encoded by the sequence as set forth in SEQ ID NOs: 31 or 32. In particular embodiments the 3′ box is encoded by the sequence as set forth in SEQ ID NOs: 33 or 34.

Particular embodiments provide artificial expression constructs including the features of vector CN4706, which include from 5′ to 3′: a DLX2.0 enhancer (SEQ ID NO: 22), a U7 promoter (SEQ ID NO: 23), a sequence encoding an miR-128 binding site (SEQ ID NO: 24), a sequence encoding an miR-221 binding site (SEQ ID NO: 25), a sequence encoding an ASO (SEQ ID NO: 30), a sequence encoding a U7 smOPT (SEQ ID NOs: 31 or 32) and a sequence encoding a mouse U7 3′box (SEQ ID NO: 33). In particular embodiments, the artificial expression construct has the sequence as set forth in SEQ ID NO: 35 or a sequence having at least 90% sequence identity to the sequence as set forth in SEQ ID NO: 35.

Particular embodiments provide artificial expression constructs including the features of vector CN4992, which include from 5′ to 3′: a DLX2.0 enhancer (SEQ ID NO: 22), a U7 promoter (SEQ ID NO: 23), a sequence encoding an ASO (SEQ ID NO: 1), a sequence encoding U7 smOPT (SEQ ID NOs: 31 or 32) and a sequence encoding a mouse U7 3′box (SEQ ID NO: 33). In particular embodiments, the artificial expression construct has the sequence as set forth in SEQ ID NO: 36 or a sequence having at least 90% sequence identity to the sequence as set forth in SEQ ID NO: 36.

Particular embodiments provide artificial expression constructs including the features of vector CN4993, which include from 5′ to 3′: a DLX2.0 enhancer (SEQ ID NO: 22), a U7 promoter (SEQ ID NO: 23), a sequence encoding an miR-128 binding site (SEQ ID NO: 24), a sequence encoding an miR-221 binding site (SEQ ID NO: 25), a sequence encoding an ASO (SEQ ID NO: 1), a sequence encoding U7 smOPT (SEQ ID NOs: 31 or 32) and a sequence encoding a mouse U7 3′box (SEQ ID NO: 33). In particular embodiments, the artificial expression construct has the sequence as set forth in SEQ ID NO: 37 or a sequence having at least 90% sequence identity to the sequence as set forth in SEQ ID NO: 37.

When artificial expression constructs disclosed herein are utilized to express disclosed ASO, the ASO are selectively expressed in inhibitory neurons providing a targeted therapeutic for patients with SCN1A-related disorders. In particular embodiments, the targeted therapeutic results in restored sodium voltage-gated channel function in inhibitory neurons.

Aspects of the disclosure are now described with the following additional options and detail: (i) Antisense Oligonucleotides (AOS); (ii) Artificial Expression Constructs & Vectors for Targeted Expression of Genes in Inhibitory Neurons; (iii) Compositions for Administration (iv) Cell Lines Including Artificial Expression Constructs; (v) Transgenic Animals; (vi) Methods of Use; (vii) Kits and Commercial Packages; (viii) Exemplary Embodiments; and (ix) Closing Paragraphs. These headings are provided for organization purposes only and do not limit the scope or interpretation of the disclosure.
(i) Antisense Oligonucleotide (ASO) s. ASO refers to an oligomer such as a polynucleotide, including a string of nucleotides that hybridize to a target nucleic acid sequence by Watson-Crick base pairing or wobble base pairing (G-U). The ASO may have exact sequence complementarity with a target sequence or near complementarity (e.g., sufficient complementarity to bind and alter the activity of a target sequence) with the target sequence. ASOs are designed so that they bind (hybridize) to a target nucleic acid (e.g., a targeted portion of a pre-mRNA transcript) and remain hybridized under physiological conditions. Typically, if they hybridize to a site other than the intended (targeted) nucleic acid sequence, they hybridize to a limited number of sequences that are not a target nucleic acid (to a few sites other than the target nucleic acid).

For example, in particular embodiments, an ASO may hybridize to an undesirable exon within an Scn1A pre-mRNA. After splicing, the Scn1a pre-mRNA hybridized to the ASO results in Scn1a mRNA without the undesirable exon.

In some embodiments, ASOs “specifically hybridize” to or are “specific” to a target nucleic acid. Typically, such hybridization occurs with a Tₘ substantially greater than 37 °C, preferably at least 50 °C, and typically between 60 °C to 90 °C. Such hybridization preferably corresponds to stringent hybridization conditions. At a given ionic strength and pH, the Tₘ is the temperature at which 50% of a target sequence hybridizes to a complementary oligonucleotide.

Oligomers, such as oligonucleotides, are “complementary” to one another when hybridization occurs in an antiparallel configuration between two single-stranded polynucleotides. A double-stranded polynucleotide can be “complementary” to another polynucleotide if hybridization can occur between one of the strands of the first polynucleotide and the second polynucleotide. Complementarity (the degree to which one polynucleotide is complementary with another) is quantifiable in terms of the proportion (e.g., the percentage) of bases in opposing strands that are expected to form hydrogen bonds with each other, according to generally accepted base-pairing rules. The sequence of an ASO need not be 100% complementary to that of its target nucleic acid to hybridize. In certain embodiments, ASOs can include at least 70%, at least 75%, at least 80%, at least 85%, at least 90%, at least 95%, at least 96%, at least 97%, at least 98%, or at least 99% sequence complementarity to a target region within the target nucleic acid sequence to which they are targeted. For example, an ASO has 90% complementarity if 18 of 20 nucleotides of the oligomeric compound are complementary to a target sequence. In this example, remaining non-complementary nucleotides may be clustered together or interspersed with complementary nucleotides and need not be contiguous to each other or to complementary nucleotides. Percent complementarity of an ASO with a region of a target nucleic acid can be determined routinely using BLAST programs (basic local alignment search tools) and PowerBLAST programs known in the art (Altschul, et al., J. Mol. Biol., 1990, 215:403-410; and Zhang and Madden, Genome Res., 1997, 7:649-656).

An ASO need not hybridize to all nucleotides in a target sequence and the nucleotides to which it does hybridize may be contiguous or noncontiguous. An ASO may hybridize over one or more segments of a sequence, such that intervening or adjacent segments are not involved in the hybridization event (e.g., a loop structure or hairpin structure may be formed). In certain embodiments, an ASO hybridizes to noncontiguous nucleotides.

The ASOs described herein include nucleotides that are complementary to nucleotides present 5′ proximal to the primary open reading frame (PORF) ATG or within a 5′ UTR exon from the 1a transcription start site (TSS) or 1b TSS. The ASOs may include naturally-occurring nucleotides, nucleotide analogs, modified nucleotides, or any combination of two or three of the preceding. The term “naturally occurring nucleotides” includes deoxyribonucleotides and ribonucleotides. The term “modified nucleotides” includes nucleotides with modified or substituted sugar groups and/or having a modified backbone. In some embodiments, all of the nucleotides of the ASO are modified nucleotides. Chemical modifications of ASOs or components of ASOs that are compatible with the methods and compositions described herein will be evident to one of skill in the art and can be found, for example, in U.S. Pat. Nos. 8,258,109; 5,656,612; US 2012/0190728, and Dias and Stein, Mol. Cancer Ther. 2002, 347-355.

The ASOs described herein also include a backbone structure that connects the nucleotides of an oligomer. The term “backbone structure” and “internucleotide linkages” may be used interchangeably and refer to the connection between monomers of the ASO. In naturally occurring oligonucleotides, the backbone includes a 3′-5′ phosphodiester linkage connecting sugar moieties of the oligomer. The backbone structure or oligomer linkages of the ASOs described herein may include phosphorothioate, phosphorodithioate, phosphoroselenoate, phosphorodiselenoate, phosphoroanilothioate, phosphoraniladate, phosphoramidate, and the like. See, e.g., LaPlanche, et al., Nucleic Acids Res. 14:9081 (1986); Stec, et al., J. Am. Chem. Soc. 106:6077 (1984), Stein, et al., Nucleic Acids Res. 16:3209 (1988), Zon, et al., Anti-Cancer Drug Design 6:539 (1991); Zon, et al., Oligonucleotides and Analogues: A Practical Approach, pp. 87-108 (F. Eckstein, Ed., Oxford University Press, Oxford England (1991)); Stec, et al., U.S. Pat. No. 5,151,510; Uhlmann and Peyman, Chemical Reviews 90:543 (1990). In some embodiments, the backbone structure of the ASO does not contain phosphorous but rather contains peptide bonds, for example in a peptide nucleic acid (PNA), or linking groups including carbamate, amides, and linear and cyclic hydrocarbon groups. In some embodiments, the backbone modification is a phosphothioate linkage. In some embodiments, the backbone modification is a phosphoramidate linkage. Some embodiments of the ASO include phosphorothioate (PS) internucleotide linkages.

In some embodiments, the stereochemistry at each of the internucleotide linkages of the ASO backbone is random. In particular embodiments, the stereochemistry at each of the internucleotide linkages of the ASO backbone is controlled and is not random. For example, US 2014/0194610, “Methods for the Synthesis of Functionalized Nucleic Acids,” describes methods for independently selecting the handedness or chirality at each phosphorous atom in a nucleic acid oligomer. In particular embodiments, an ASO includes phosphorus internucleotide linkages that are not random. In particular embodiments, a composition used in the methods of the disclosure includes a pure diastereomeric ASO. In particular embodiments, a composition used in the methods of the disclosure includes an ASO that has diastereomeric purity of at least 90%, at least 91%, at least 92%, at least 93%, at least 94%, at least 95%, at least 96%, at least 97%, at least 98%, at least 99%, or of 100%. In particular embodiments, a composition used in the methods of the disclosure includes an ASO that has diastereomeric purity of 90% to 100%, 91% to 100%, 92% to 100%, 93% to 100%, 94% to 100%, 95% to 100%, 96% to 100%, 97% to 100%, 98% to 100%, or 99% to 100%.

In particular embodiments, the ASO has a nonrandom mixture of R^{p} and Sp configurations at its phosphorus internucleotide linkages. In particular embodiments, an ASO used in the methods of the disclosure, including any of the ASOs set forth herein in SEQ ID NOs: 1-12 or 30, includes 5-100% Rp, at least 5% Rp, at least 10% Rp, at least 15% Rp, at least 20% Rp, at least 25% Rp, at least 30% Rp, at least 35% Rp, at least 40% Rp, at least 45% Rp, at least 50% Rp, at least 55% Rp, at least 60% Rp, at least 65% Rp, at least 70% Rp, at least 75% Rp, at least 80% Rp, at least 85% Rp, at least 90% Rp, or at least 95% Rp, with the remainder Sp, or 100% Rp. In particular embodiments, an ASO used in the methods of the disclosure, including any of the ASOs set forth herein in SEQ ID NOs: 1-12 or 30, includes 10% to 100% Rp, 15% to 100% Rp, 20% to 100% Rp, 25% to 100% Rp, 30% to 100% Rp, 35% to 100% Rp, 40% to 100% Rp, 45% to 100% Rp, 50% to 100% Rp, 55% to 100% Rp, 60% to 100% Rp, 65% to 100% Rp, 70% to 100% Rp, 75% to 100% Rp, 80% to 100% Rp, 85% to 100% Rp, 90% to 100% Rp, or 95% to 100% Rp, 20% to 80% Rp, 25% to 75% Rp, 30% to 70% Rp, 40% to 60% Rp, or 45% to 55% Rp, with the remainder Sp.

Any of the ASOs described herein may contain a sugar moiety that includes ribose or deoxyribose, as present in naturally occurring nucleotides, or a modified sugar moiety or sugar analog, including a morpholine ring. Examples of modified sugar moieties include 2′ substitutions such as 2′-O-methoxyethyl (2′MOE), 2′-O-methyl(2′-O-Me), 2′-O-aminoethyl, 2′F; N3′->P5′ phosphoramidate, 2′dimethylaminooxyethoxy, 2′dimethylaminoethoxyethoxy, 2′-guanidinidium, 2′-O-guanidinium ethyl, carbamate modified sugars, and bicyclic modified sugars. In some embodiments, the sugar moiety modification is selected from 2′-O-Me, 2′F, and 2′MOE. In some embodiments, the sugar moiety modification is an extra bridge bond, such as in a locked nucleic acid (LNA). In some embodiments the sugar analog contains a morpholine ring, such as phosphorodiamidate morpholino (PMO). In some embodiments, the sugar moiety includes a ribofuransyl or 2′deoxyribofuransyl modification. In some embodiments, the sugar moiety includes 2′4′-constrained 2′O-methyloxyethyl (cMOE) modifications. In some embodiments, the sugar moiety includes cEt 2′, 4′ constrained 2′-O ethyl BNA modifications. In some embodiments, the sugar moiety includes tricycloDNA (tcDNA) modifications. In some embodiments, the sugar moiety includes ethylene nucleic acid (ENA) modifications. In some embodiments, the sugar moiety includes MCE modifications. Modifications are known in the art and described in the literature, e.g., by Jarver, et al., 2014, “A Chemical View of Oligonucleotides for Exon Skipping and Related Drug Applications,” Nucleic Acid Therapeutics 24 (1): 37-47.

In some embodiments, each monomer of the ASO is modified in the same way, for example each linkage of the backbone of the ASO includes a phosphorothioate linkage or each ribose sugar moiety includes a 2′O-methyl modification. Such modifications that are present on each of the monomer components of an ASO are referred to as “uniform modifications.” In some examples, a combination of different modifications may be desired, for example, an ASO may include a combination of phosphorodiamidate linkages and sugar moieties including morpholine rings (morpholinos). Combinations of different modifications to an ASO are referred to as “mixed modifications” or “mixed chemistries.”

In some embodiments, the ASO includes one or more backbone modifications. In some embodiments, the ASO includes one or more sugar moiety modifications. In some embodiments, the ASO includes one or more backbone modifications and one or more sugar moiety modifications. In some embodiments, the ASO includes a 2′MOE modification and a phosphorothioate backbone. In some embodiments, the ASO includes a phosphorodiamidate morpholino (PMO). In some embodiments, the ASO includes a peptide nucleic acid (PNA). Any of the ASOs or any component of an ASO (e.g., a nucleotide, sugar moiety, backbone) described herein may be modified in order to achieve desired properties or activities of the ASO or reduce undesired properties or activities of the ASO. For example, an ASO or one or more components of any ASO may be modified to enhance binding affinity to a target sequence; reduce binding to any non-target sequence; reduce degradation by cellular nucleases (i.e., RNase H); improve uptake of the ASO into a cell and/or into the nucleus of a cell; alter the pharmacokinetics or pharmacodynamics of the ASO; and/or modulate the half-life of the ASO.

In some embodiments, the ASO includes 2′-O-(2-methoxyethyl) (2′-MOE) bases and phosphorothioate-internucleoside linkages. ASOs including such nucleotides are especially well-suited to the methods disclosed herein; oligomers having such modifications have been shown to have significantly enhanced resistance to nuclease degradation and increased bioavailability, making them suitable, for example, for oral delivery in some embodiments described herein. See e.g., Geary, et al., J Pharmacol Exp Ther. 2001; 296 (3): 890-7; Geary, et al., J Pharmacol Exp Ther. 2001; 296 (3): 898-904.

Methods of synthesizing ASOs will be known to one of skill in the art. Alternatively or in addition, ASOs may be obtained from a commercial source.

The ASO may be of any length suitable for specific binding. In some embodiments, the ASO includes 8 to 50 nucleotides. For example, the ASO may be 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, or 50 nucleotides in length. In some embodiments, the ASO includes more than 50 nucleotides. In some embodiments, the ASO is from 8 to 50 nucleotides, 8 to 40 nucleotides, 8 to 35 nucleotides, 8 to 30 nucleotides, 8 to 25 nucleotides, 8 to 20 nucleotides, 8 to 15 nucleotides, 9 to 50 nucleotides, 9 to 40 nucleotides, 9 to 35 nucleotides, 9 to 30 nucleotides, 9 to 25 nucleotides, 9 to 20 nucleotides, 9 to 15 nucleotides, 10 to 50 nucleotides, 10 to 40 nucleotides, 10 to 35 nucleotides, 10 to 30 nucleotides, 10 to 25 nucleotides, 10 to 20 nucleotides, 10 to 15 nucleotides, 11 to 50 nucleotides, 11 to 40 nucleotides, 11 to 35 nucleotides, 11 to 30 nucleotides, 11 to 25 nucleotides, 11 to 20 nucleotides, 11 to 15 nucleotides, 12 to 50 nucleotides, 12 to 40 nucleotides, 12 to 35 nucleotides, 12 to 30 nucleotides, 12 to 25 nucleotides, 12 to 20 nucleotides, 12 to 15 nucleotides, 13 to 50 nucleotides, 13 to 40 nucleotides, 13 to 35 nucleotides, 13 to 30 nucleotides, 13 to 25 nucleotides, 13 to 20 nucleotides, 14 to 50 nucleotides, 14 to 40 nucleotides, 14 to 35 nucleotides, 14 to 30 nucleotides, 14 to 25 nucleotides, 14 to 20 nucleotides, 15 to 50 nucleotides, 15 to 40 nucleotides, 15 to 35 nucleotides, 15 to 30 nucleotides, 15 to 25 nucleotides, 15 to 20 nucleotides, 20 to 50 nucleotides, 20 to 40 nucleotides, 20 to 35 nucleotides, 20 to 30 nucleotides, 20 to 25 nucleotides, 25 to 50 nucleotides, 25 to 40 nucleotides, 25 to 35 nucleotides, or 25 to 30 nucleotides in length. In some embodiments, the ASO is 12-24 nucleotides in length. In some embodiments, the ASO is 15-21 nucleotides in length. In some embodiments, the ASO is 18 nucleotides in length.

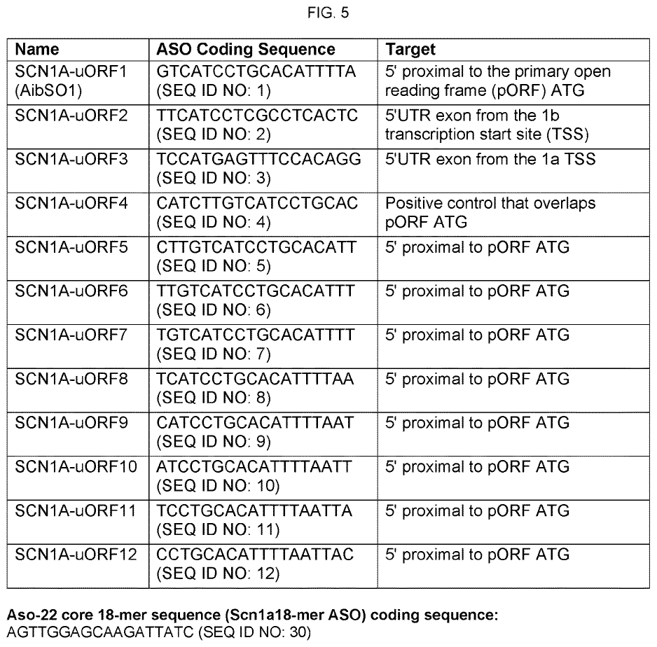

Particular examples of ASO coding sequences disclosed herein are provided in FIG. 5.

The ASO provided as SEQ ID NOs: 1-12 or 30 can include 2′-O-methoxyethyl base (2′-MOE)+phosphorothioate (PS) internucleotide linkages. In particular embodiments, the ASO are provided in cyclic form.

In particular embodiments, ASO can be administered with one or more agents capable of promoting penetration of the ASO across the blood-brain barrier by any method known in the art.

In particular embodiments, the ASO can be linked or conjugated with agents that provide desirable pharmaceutical or pharmacodynamic properties. In particular embodiments, the ASO can be coupled to a substance, known in the art to promote penetration or transport across the blood-brain barrier, e.g., an antibody to the transferrin receptor. In particular embodiments, the ASO can be linked with a viral vector, e.g., to render the ASO more effective or increase transport across the blood-brain barrier. In particular embodiments, osmotic blood brain barrier disruption is assisted by infusion of sugars, e.g., meso erythritol, xylitol, D(+) galactose, D(+) lactose, D(+) xylose, dulcitol, myo-inositol, L(−) fructose, D(−) mannitol, D(+) glucose, D(+) arabinose, D(−) arabinose, cellobiose, D(+) maltose, D(+) raffinose, L(+) rhamnose, D(+) melibiose, D(−) ribose, adonitol, D(+) arabitol, L(−) arabitol, D(+) fucose, L(−) fucose, D(−) lyxose, L(+) lyxose, and L(−) lyxose, or amino acids, e.g., glutamine, lysine, arginine, asparagine, aspartic acid, cysteine, glutamic acid, glycine, histidine, leucine, methionine, phenylalanine, proline, serine, threonine, tyrosine, valine, and taurine. Methods and materials for enhancing blood brain barrier penetration are described, e.g., in U.S. Pat. No. 9,193,969, “Compositions and methods for selective delivery of oligonucleotide molecules to specific neuron types,” U.S. Pat. No. 4,866,042, “Method for the delivery of genetic material across the blood brain barrier,” U.S. Pat. No. 6,294,520, “Material for passage through the blood-brain barrier,” and U.S. Pat. No. 6,936,589, “Parenteral delivery systems.”

In some embodiments, the ASO described herein are used to increase the production of a functional SCN1A protein (i.e., Nav1.1). As used herein, the term “functional” refers to the amount of activity or function of a SCN1A protein that is necessary to eliminate any one or more symptoms of a treated condition, e.g., Dravet syndrome or other SCN1A-related disorders described elsewhere herein.

In some embodiments, the level of SCN1A protein expressed by a cell including an ASO disclosed herein is increased 1.1 to 10-fold, when compared to the amount of SCN1A protein that is produced in a control cell, e.g., one that does not include an ASO disclosed herein.

In some embodiments, contacting cells with an ASO disclosed herein results in an increase in the amount of SCN1A protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of SCN1A protein produced by the cell to which the ASO is contacted is increased 1.1 to 10-fold, 1.5 to 10-fold, 2 to 10-fold, 3 to 10-fold, 4 to 10-fold, 1.1 to 5-fold, 1.1 to 6-fold, 1.1 to 7-fold, 1.1 to 8-fold, 1.1 to 9-fold, 2 to 5-fold, 2 to 6-fold, 2 to 7-fold, 2 to 8-fold, 2 to 9-fold, 3 to 6-fold, 3 to 7-fold, 3 to 8-fold, 3 to 9-fold, 4 to 7-fold, 4 to 8-fold, or 4 to 9-fold, compared to a reference level. In some embodiments, the total amount of SCN1A protein produced by the cell to which the ASO is contacted is increased by at least 1.1-fold, at least 1.5-fold, at least 2-fold, at least 2.5-fold, at least 3-fold, at least 3.5-fold, at least 4-fold, at least 5-fold, or at least 10-fold, compared to a reference level. In particular embodiments, the reference level is the amount of target protein produced by a control ASO. A control ASO can be, for example, the ASO as set forth in SEQ ID NO: 4. In particular embodiments, the reference level is the amount of target protein produced by a control artificial expression construct without an ASO.

In some embodiments, an ASO is used to increase the functional expression of SCN1A protein in cells of a subject having pre-mRNA encoding SCN1A protein, wherein the subject has a deficiency in the amount or function of a SCN1A protein (i.e., SNC1A-related disorder). Examples of SCN1A-related disorders include: Dravet Syndrome (DS); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); sick sinus syndrome 1; early infantile SCN1A encephalopathy; early infantile epileptic encephalopathy (EIEE); autism; hemiplegic migraine; arthrogryposis multiplex congenita (AMC); myoclonic-atonic epilepsy (MAE); epilepsy of infancy with migrating focal seizures (EIMFS); Rett syndrome; nonsyndromic epileptic encephalopathy (NEE); or intractable childhood epilepsy with generalized tonic-clonic seizures (ICE-GTC).
(ii) Artificial Expression Constructs & Vectors for Targeted Expression of RNA in Inhibitory Neurons. Artificial expression constructs disclosed herein include or encode:
(i) an enhancer, (ii) a U7 promoter; (iii) an ASO, (iv) an Sm binding domain, and (iv) a 3′ box; or (i) an enhancer, (ii) a U7 promoter; (iii) an miR binding site; (iv) an ASO, (v) an Sm binding domain, and (v) a 3′ box.
   The artificial expression construct can include or encode other regulatory elements if necessary or beneficial.

In particular embodiments, an “enhancer” or an “enhancer element” is a cis-acting sequence that increases the level of transcription associated with a promoter and can function in either orientation relative to the promoter and a coding or non-coding sequence and can be located upstream or downstream relative to the promoter or the coding or non-coding sequence. There are art-recognized methods and techniques for measuring function(s) of enhancers. Particular examples of enhancers utilized within artificial expression constructs disclosed herein include hl56i (core) and DLX2.0.

Promoters can include general promoters, tissue-specific promoters, cell-specific promoters, and/or promoters specific for the cytoplasm. Promoters may include strong promoters, weak promoters, constitutive expression promoters, and/or inducible promoters. Inducible promoters direct expression in response to certain conditions, signals or cellular events. For example, the promoter may be an inducible promoter that requires a particular ligand, small molecule, transcription factor or hormone protein in order to effect transcription from the promoter. Particular examples of promoters include the U7 promoter (U7prom), minBglobin (also referred to as minBGprom), CMV, minCMV, minCMV* (minCMV* is minCMV with a Sacl restriction site removed), minRho, minRho* (minRho* is minRho with a Sacl restriction site removed), SV40 immediately early promoter, the Hsp68 minimal promoter (proHSP68), and the Rous Sarcoma Virus (RSV) long-terminal repeat (LTR) promoter. Minimal promoters have no activity to drive expression on their own but can be activated to drive expression when linked to an enhancer element. In particular embodiments, the minimal promoter can be activated to drive expression when linked to a proximal enhancer element. In particular embodiments, the promoter includes a U7 promoter. In particular embodiments, the promoter includes a U7promDel2 promoter which is a U7 promoter having a deletion.

A microRNA (“miRNA”) is a small non-coding RNA molecule that functions in RNA silencing and post-transcriptional regulation of gene expression via base pairing with complementary sequences within messenger RNA (mRNA) molecules. Under the standard nomenclature system, the prefix “miR” is followed by a dash and a number. “miR-” refers to the mature form of the miRNA; “mir-” refers to the primary mRNA (pri-miRNA) or the precursor miRNA (pre-miRNA); and “MIR” refers to the gene that encodes them. miRNAs with nearly identical sequences are annotated with an additional lower case letter. Species of origin is designated with a three-letter prefix, e.g., “hsa-” for human. miRNA genes that lead to identical mature miRNAs, but are located at different places in the genome, are indicated with an additional dash-number suffix, e.g., miR-194-1 and miR-194-2. Native human miRNAs are typically transcribed as the >100 nucleotide pri-miRNA, which is processed to form the pre-miRNA, which is further processed to form the mature miRNA. Exemplary miR bindings sites include miR-128 binding site, miR-221 binding site, miR-222 binding site, miR-133b binding site, miR-187 binding site, miR-138 binding site, and miR-335-5p miRNA binding site. In particular embodiment, an miR binding site can include multiple miRNA binding sites such as in 1x2C, 4x2C. or 8x2C. In particular embodiments, 1x2C includes an miR-128 binding site and an miR-221 binding site. In particular embodiments, 1x2C includes a sequence as set forth in any of SEQ ID NOs: 26-29. In particular embodiments, 4x2C includes 4 miR-128 binding sites and 4 miR-221 binding sites. In particular embodiments, 4x2C includes a sequence as set forth in SEQ ID NO: 44. In particular embodiments, 8x2C includes 8 miR-128 binding sites and 8 miR-221 binding sites. In particular embodiments, 8x2C includes a sequence as set forth in SEQ ID NO: 43.

Artificial expression constructs disclosed herein include an miR binding site coding sequence between a U7 promoter and an ASO coding sequence. miR binding sites can promote specific repression of expression of one or more ASOs in cell types that are not GABAergic (i.e., inhibitory) neurons.

Artificial expression constructs disclosed herein can also include an ASO coding sequence having the sequence as set forth in any of SEQ ID NOs: 1-12 or 30.

Small nuclear RNA (snRNA) is a class of small RNA molecules that are found within the splicing speckles and Cajal bodies of the cell nucleus in eukaryotic cells. snRNA are often divided into two classes: the Sm-class snRNA and the Lsm-class snRNA. The Sm-class snRNA genes are transcribed by RNA polymerase II in association with a specialized complex called Integrator. The polymerase initiate transcription on short bipartite promoters composed of proximal and distal sequence elements (PSE and DSE, respectively), while the Integrator complex recognizes signals in the internal stem-loop structures of the sequence, as well as in a conserved 3′ box and mediates co-transcriptional 3′ processing (Baillat et al., Cell. 2005, 123:265-276). The snRNAs are then exported to the cytoplasm where they undergo further modifications and assemble with Sm proteins before re-entry into the nucleus. Proper assembly requires a consensus Sm binding site (also referred to as Sm binding domain) (Patel and Bellini, Nucleic Acids Res. 2008, 36:6482-6493). U7 is a non-spliceosomal snRNA that functions as a processing factor of the non-polyadenylated replication dependent histone mRNA. It is the shortest and simplest of snRNAs and it includes a 5′ ASO recognizing the histone pre-mRNA, a non-consensus Sm-binding site and a stem-loop structure containing determinants for the co-transcriptional 3′ endonucleolytic cleavage. The low abundance of the U7 small nuclear ribonucleoproteins (snRNP) is reversed when a consensus spliceosomal Sm sequence is introduced (SmOPT) and substitutions of the original 5′ sequences result in abundant and stable shuttles for ectopic antisense delivery (Gorman et al., Proc Natl Acad Sci USA. 1998, 95:4929-4934; and Grimm et al., EMBO J. 1993; 12:1229-1238).

In particular embodiments, the artificial expression construct includes an Sm binding domain coding sequence. Sm and Sm-like proteins are members of a family of small proteins that is widespread throughout eukaryotic kingdoms. The seven human Sm proteins include B, D1, D2, D3, E, F, and G. As described above, the Sm proteins assemble at the Sm binding domain. The Sm binding domain that is present on snRNAs follows the consensus sequence RAU5GR, wherein R is any purine (Branlant et al., EMBO J. 1982; 1:1259-1265). In particular embodiments, the Sm binding domain is SmOpt. In particular embodiments, SmOpt coding sequence includes the sequence as set forth in SEQ ID NOs: 31 or 32.

In particular embodiments, the artificial expression construct includes a 3′ box coding sequence. The 3′ end of snRNA genes contains a 3′-box required for proper 3′ end formation located 9-28 nts downstream of the snRNA-coding region (Hernandez. EMBO J. 1985, 4:1827-1837; and Ach and Weiner Mol Cell Biol. 1987, 7:2070-2079). The 3′ box is likely to be a recognition site for a complex of proteins required to cleave the nascent snRNA from the gene. Once the snRNA is cleaved off the template, it undergoes export to the cytoplasm through the activity of the snRNA-specific export factor PHAX (phosphorylated adapter for RNA export) and then continues onward in the snRNP (small nuclear ribonucleoprotein) biogenesis pathway (Chen and Wagner. Biochem Soc Trans 2010, 38 (4): 1082-1087).

In particular embodiments, the artificial expression construct encodes a hairpin loop. A hairpin loop, also known as a stem-loop structure, is a particular pattern found in single-stranded DNA or RNA where the sequence of nucleotides is such that it can loop back on itself, forming a structure resembling a hairpin or a staple.

In a hairpin loop, the strand loops out and then comes back to pair with itself, forming a stem ending in an unpaired loop. This structure is thermodynamically stable and is held together by the hydrogen bonding of base pairs. It is typically formed in sequences where consecutive nucleotides are complementary to each other. Hairpin loops play important roles in the regulation of genetic expression and have been found to have crucial functions in viral replication, regulation of gene expression, and molecular recognition in biological systems. They are often targets for enzymes and can be manipulated for the design of new therapeutics.

In particular embodiments, the hairpin loop length is 7-15 nucleotides, such as 7 nucleotides, 8 nucleotides, 9 nucleotides, 10 nucleotides, 11 nucleotides, 12, nucleotides, 13 nucleotides, 14 nucleotides, or 15 nucleotides. If a given nucleic acid sequence is characterized by a hairpin loop, the respective complementary nucleic acid sequence is typically also characterized by a hairpin loop. A hairpin loop is typically formed by single-stranded RNA molecules. In particular embodiments, the hairpin loop length is at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, or at least 23 nucleotides in length.

Hairpin loops are often present within 3′ UTR of highly expressed transcripts. The hairpin loops associate with stem loop binding proteins (SLBPs) for replication-dependent RNA stability/processing/metabolism/translation. Structural evidence suggests that the direct contact of SLBPs with hairpin loops occurs at a guanosine nucleotide at the base of SL (G7) (Tan et al., Science (80). (2013); Battle & Doudna, doi: 10.1017/S1355838201001820).

In particular embodiments, the sequence encoding the hairpin loop is between the smOPT coding sequence and the 3′box coding sequence. In particular embodiments, the U7 smOPT coding sequence includes the sequence encoding the hairpin loop. In particular embodiments, 3′box coding sequence includes the sequence encoding the hairpin loop. In particular embodiments, the includes the hairpin loop sequence: CAGGTTTTCTGACTTCGGTCGGAAAACCCCT (SEQ ID NO: 52). In particular embodiments, hairpin loops are not sequence-orientation dependent and may include a) CCUC and GAGG, b) GAGG and CCUC, c) CUCC and GGAG, or d) GGAG and CUCC. Further, the distance between the two arms of the stem (where the CCUC and GAGG base pair) needs to be long enough for a loop to form. In particular embodiments, stem loops can include complementary sequences such as a) RRRR and YYYY, b) RYRR and YRYY, c) RRYR and YYRY, d) RRRY and YYYR, e) RYYR and YRRY, f) RRYY and YYRR, g) YYRR and RRYY, h) YYYR and RRRY, or i) RYYY and YRRR, wherein R is a purine (A or G) and Y is a pyrimidine (e.g., U or C).

The term “encode” or “encoding” refers to a property of sequences of nucleic acids, such as a vector, a plasmid, a gene, cDNA, mRNA, to serve as templates for synthesis of other molecules such as RNA or proteins. In particular embodiments, an artificial expression construct encodes an RNA sequence. In particular embodiments, a coding sequence includes a DNA sequence that encodes RNA.

In particular embodiments, artificial expression constructs are provided within vectors. The term vector refers to a nucleic acid molecule capable of transferring or transporting another nucleic acid molecule, such as an artificial expression construct. The transferred nucleic acid is generally linked to, e.g., inserted into, the vector nucleic acid molecule. A vector may include sequences that direct autonomous replication in a cell or may include sequences that permit integration into host cell DNA. Useful vectors include, for example, plasmids (e.g., DNA plasmids or RNA plasmids), transposons, cosmids, bacterial artificial chromosomes, and viral vectors.

“Viral vector” is widely used to refer to a nucleic acid molecule that includes virus-derived components that facilitate transfer and expression of non-native nucleic acid molecules within a cell. The term adeno-associated viral vector refers to a viral vector or plasmid containing structural and functional genetic elements, or portions thereof, that are primarily derived from an adeno-associate virus (AAV). The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, that are primarily derived from a lentivirus, and so on. The term “hybrid vector” refers to a vector including structural and/or functional genetic elements from more than one virus type.

Adeno-Associated Virus (AAV) is a parvovirus, discovered as a contamination of adenoviral stocks. It is a ubiquitous virus (antibodies are present in 85% of the US human population) that has not been linked to any disease. It is also classified as a dependovirus because its replication is dependent on the presence of a helper virus, such as an adenovirus. Various serotypes have been isolated, of which AAV-2 is the best characterized. AAV has a single-stranded linear DNA that is encapsidated into capsid proteins VP1, VP2 and VP3 to form an icosahedral virion of 20 to 24 nm in diameter.

The AAV DNA is 4.7 kilobases long. It contains two open reading frames and is flanked by two ITRs. There are two major genes in the AAV genome: rep and cap. The rep gene codes for proteins responsible for viral replications, whereas cap codes for capsid protein VP1-3. Each ITR forms a T-shaped hairpin structure. These terminal repeats are the only essential cis components of the AAV for chromosomal integration. Therefore, the AAV can be used as a vector with all viral coding sequences removed and replaced by the cassette of genes for delivery. Three AAV viral promoters have been identified and named p5, p19, and p40, according to their map position. Transcription from p5 and p19 results in production of rep proteins, and transcription from p40 produces the capsid proteins.

AAVs stand out for use within the current disclosure because of their superb safety profile and because their capsids and genomes can be tailored to allow expression in targeted cell populations. scAAV refers to a self-complementary AAV. pAAV refers to a plasmid adeno-associated virus. rAAV refers to a recombinant adeno-associated virus.

Other viral vectors may also be employed. For example, vectors derived from viruses such as vaccinia virus, polioviruses and herpes viruses may be employed. They offer several attractive features for various mammalian cells.

Adenovirus vectors refer to those constructs containing adenovirus sequences sufficient to (a) support packaging of an artificial expression construct and (b) to express a sequence that has been cloned therein in a sense or antisense orientation. A recombinant adenovirus vector includes a genetically engineered form of an adenovirus. Knowledge of the genetic organization of adenovirus, a 36 kb, linear, double-stranded DNA virus, allows substitution of large pieces of adenoviral DNA with foreign sequences up to 7 kb. In contrast to a retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range, and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut-off. The products of the late genes, including the majority of the viral capsid proteins, are expressed only after significant processing of a single primary transcript issued by the major late promoter (MLP). The MLP is particularly efficient during the late phase of infection, and all the mRNAs issued from this promoter possess a 5′-tripartite leader (TPL) sequence which makes them preferred mRNAs for translation.

Other than the requirement that an adenovirus vector be replication defective, or at least conditionally defective, the nature of the adenovirus vector is not believed to be crucial to the successful practice of particular embodiments disclosed herein. The adenovirus may be of any of the 42 different known serotypes or subgroups A-F. In particular embodiments, adenovirus type 5 of subgroup C is the preferred starting material in order to obtain a conditional replication-defective adenovirus vector for use in particular embodiments, since Adenovirus type 5 is a human adenovirus about which a great deal of biochemical and genetic information is known, and it has historically been used for most constructions employing adenovirus as a vector.

As indicated, the typical vector is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical. The polynucleotide encoding the gene of interest may also be inserted in lieu of a deleted E3 region in E3 replacement vectors or in the E4 region where a helper cell line or helper virus complements the E4 defect.

Retroviruses are a common tool for gene delivery. “Retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus.” The provirus serves as a template for RNA polymerase II and directs the expression of RNA molecules which encode the structural proteins and enzymes needed to produce new viral particles.

Illustrative retroviruses suitable for use in particular embodiments, include: Moloney murine leukemia virus (M-MuLV), Moloney murine sarcoma virus (MOMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV), Rous Sarcoma Virus (RSV), and lentivirus.

“Lentivirus” refers to a group (or genus) of complex retroviruses. Illustrative lentiviruses include: HIV (human immunodeficiency virus; including HIV type 1, and HIV type 2); visna-maedi virus (VMV); the caprine arthritis-encephalitis virus (CAEV); equine infectious anemia virus (EIAV); feline immunodeficiency virus (FIV); bovine immune deficiency virus (BIV); and simian immunodeficiency virus (SIV). In particular embodiments, HIV based vector backbones (i.e., HIV cis-acting sequence elements) can be used.

A safety enhancement for the use of some vectors can be provided by replacing the U3 region of the 5′ LTR with a heterologous promoter to drive transcription of the viral genome during production of viral particles. Examples of heterologous promoters which can be used for this purpose include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In particular embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include one or more chemical compounds or the physiological conditions such as temperature or pH, in which the host cells are cultured. Herein, heterologous refers to having differing elements, as parts of different organisms or of the same organisms that are unlike in structure or origin.

In particular embodiments, viral vectors include a TAR element. The term “TAR” refers to the “trans-activation response” genetic element located in the R region of lentiviral LTRs. This element interacts with the lentiviral trans-activator (tat) genetic element to enhance viral replication. However, this element is not required in embodiments wherein the U3 region of the 5′ LTR is replaced by a heterologous promoter.

The “R region” refers to the region within retroviral LTRs beginning at the start of the capping group (i.e., the start of transcription) and ending immediately prior to the start of the poly(A) tract. The R region is also defined as being flanked by the U3 and U5 regions. The R region plays a role during reverse transcription in permitting the transfer of nascent DNA from one end of the genome to the other.

Beyond the foregoing description, a wide range of suitable expression vector types will be known to a person of ordinary skill in the art. Numerous vectors are commercially available, e.g., from Invitrogen, Stratagene, Clontech, etc., and are described in numerous associated guides. In particular embodiments, suitable expression vectors include any plasmid, cosmid or phage construct that is capable of supporting expression of ASOs in mammalian neurons.

Particular embodiments of vectors disclosed herein include:

| Expression | |
|---|---|
| Construct | |
| Name | Features |
| CN4706 | rAAV-DLX2.0-U7prom-1X2C-[Scn1a18-mer-ASO]-smOPT-3′box |
| CN4992 | rAAV-DLX2.0-U7prom-[AibSO1-ASO]-smOPT-3′box |
| CN4993 | rAAV-DLX2.0-U7prom-1X2C-[AibSO1-ASO]-smOPT-3′box |
| CN4872 | pAAV-U7prom-[Scn1a18-mer-ASO]-smOPT-3′box |
| CN4710 | rAAV-DLX2.0-U7prom-4X2C-[Scn1a18-mer-ASO]-smOPT-3′box |
| CN4707 | rAAV-DLX2.0-U7prom-8X2C-[Scn1a18-mer-ASO]-smOPT-3′box |
| CN4705 | rAAV-DLX2.0-U7prom-[Scn1a18-mer-ASO]-miR128-smOPT-3′box |
| CN4708 | rAAV-DLX2.0-U7prom-[Scn1a18-mer-ASO]-smOPT-8X2C-3′box |
| CN4709 | rAAV-DLX2.0-U7prom-[Scn1a18-mer-ASO]-smOPT-3′box-8X2C |
| CN4769 | rAAV-DLX2.0-U7promDel2-[Scn1a18-mer-ASO]-smOPT-3′box |

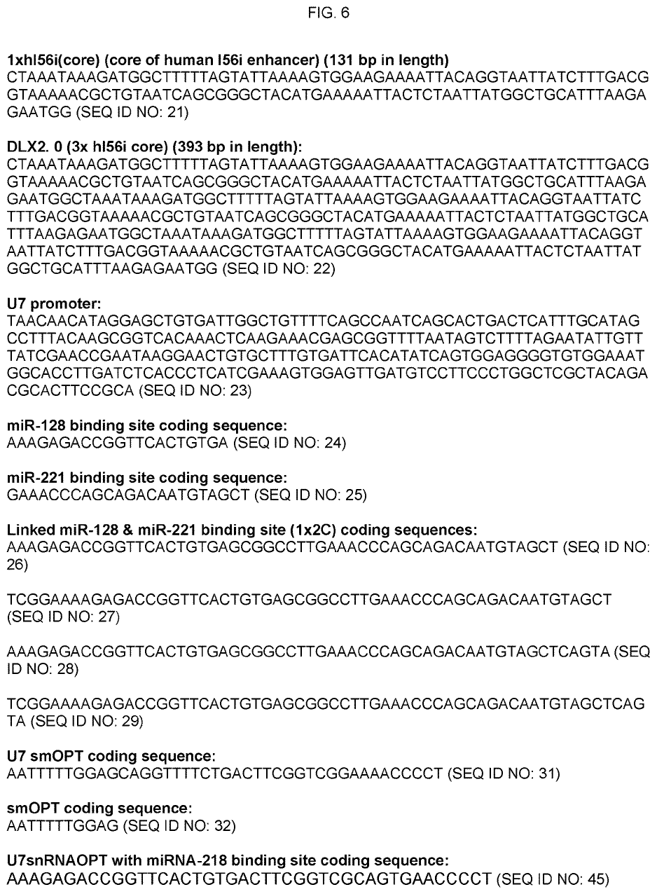

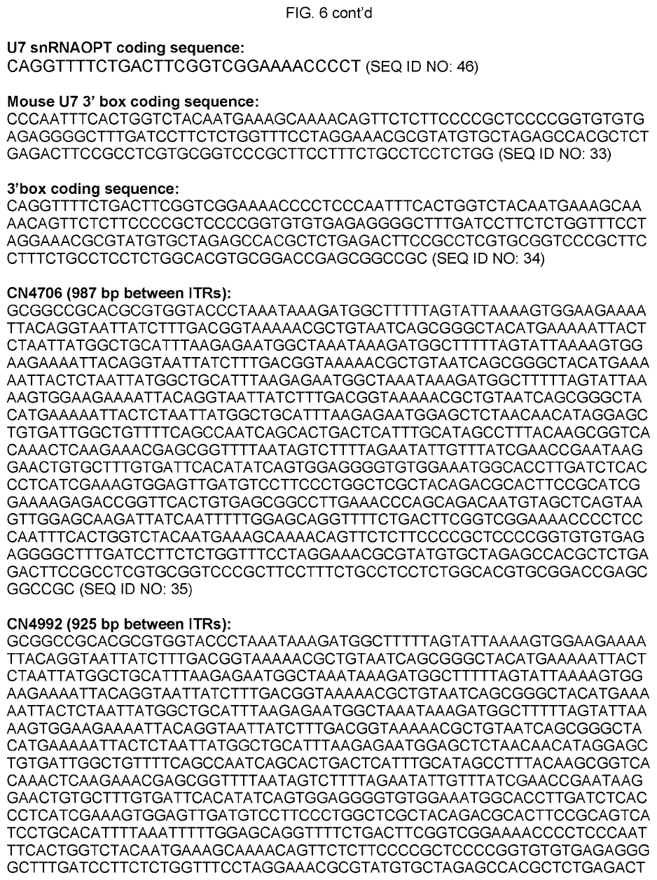

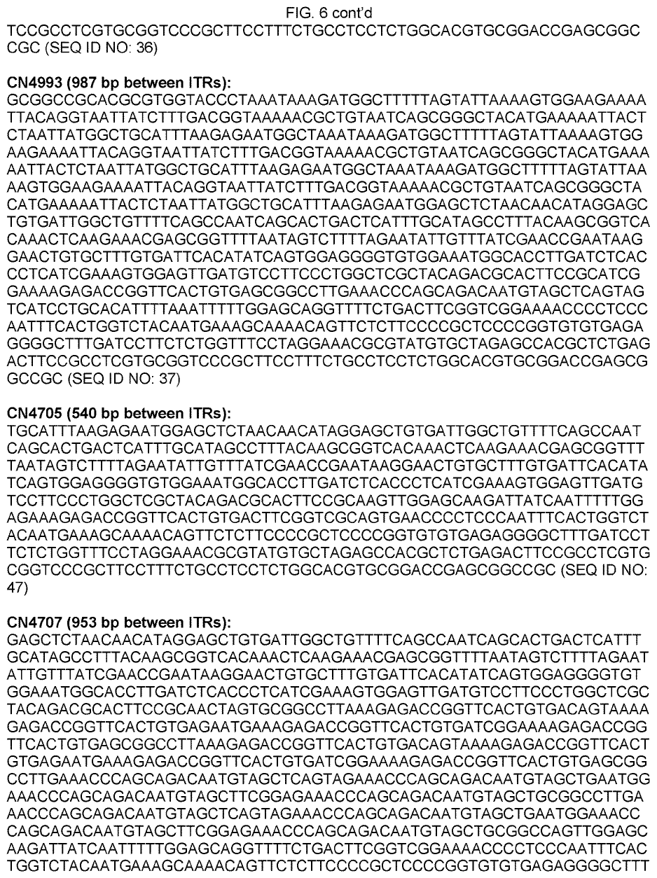

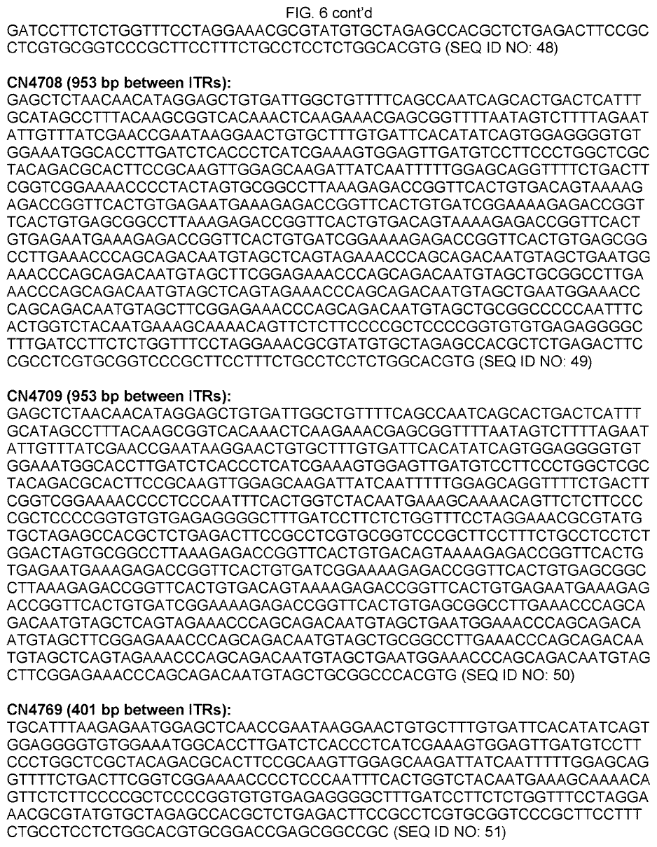

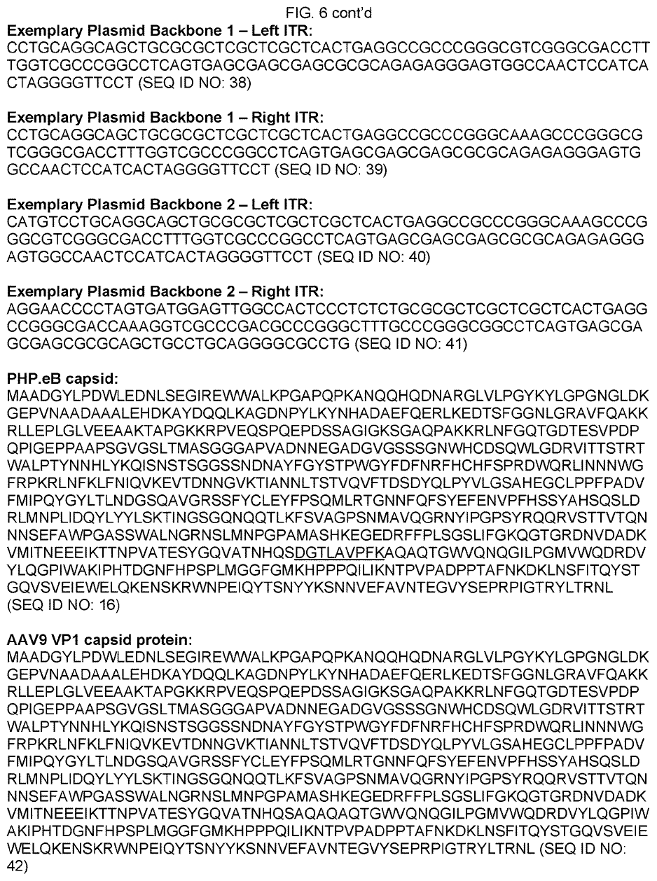

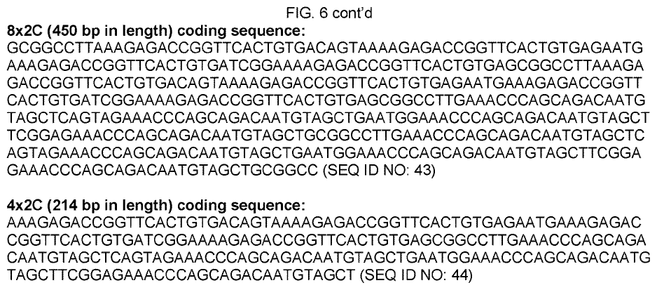

Subcomponent sequences within the larger vector sequences can be readily identified by one of ordinary skill in the art and based on the contents of the current disclosure (see FIG. 6). Nucleotides between identifiable and enumerated subcomponents reflect restriction enzyme recognition sites used in assembly (cloning) of the constructs, and in some cases, additional nucleotides do not convey any identifiable function. These segments of complete vector sequences can be adjusted based on use of different cloning strategies and/or vectors. In general, short 6-nucleotide palindromic sequences reflect vector construction artifacts that are not important to vector function.

In particular embodiments vectors (e.g., AAV) with capsids that cross the blood-brain barrier (BBB) are selected. In particular embodiments, vectors are modified to include capsids that cross the BBB. Examples of AAV with viral capsids that cross the blood brain barrier include AAV9 (Gombash et al., Front Mol Neurosci. 2014; 7:81), AAVrh. 10 (Yang, et al., Mol Ther. 2014; 22 (7): 1299-1309), AAV1R6, AAV1R7 (Albright et al., Mol Ther. 2018; 26 (2): 510), rAAVrh.8 (Yang, et al., supra), AAV-BR1 (Marchio et al., EMBO Mol Med. 2016; 8 (6): 592), AAV-PHP.S (Chan et al., Nat Neurosci. 2017; 20 (8): 1172), AAV-PHP.B (Deverman et al., Nat Biotechnol. 2016; 34 (2): 204), AAV-PPS (Chen et al., Nat Med. 2009; 15:1215), and PHP.eB. In particular embodiments, the PHP.eB capsid differs from AAV9 such that, using AAV9 as a reference, amino acids starting at residue 586: S-AQ-A (SEQ ID NO: 14) are changed to S-DGTLAVPFK-A (SEQ ID NO: 15). In particular embodiments, PHP.eb refers to SEQ ID NO: 16.

AAV9 is a naturally occurring AAV serotype that, unlike many other naturally occurring serotypes, can cross the BBB following intravenous injection. It transduces large sections of the central nervous system (CNS), thus permitting minimally invasive treatments (Naso et al., BioDrugs. 2017; 31 (4): 317), for example, as described in relation to clinical trials for the treatment of spinal muscular atrophy (SMA) syndrome by AveXis (AVXS-101, NCT03505099) and the treatment of CLN3 gene-Related Neuronal Ceroid-Lipofuscinosis (NCT03770572).

AAVrh. 10, was originally isolated from rhesus macaques and shows low seropositivity in humans when compared with other common serotypes used for gene delivery applications (Selot et al., Front Pharmacol. 2017; 8:441) and has been evaluated in clinical trials LYS-SAF302, LYSOGENE, and NCT03612869.

AAV1R6 and AAV1R7, two variants isolated from a library of chimeric AAV vectors (AAV1 capsid domains swapped into AAVrh. 10), retain the ability to cross the BBB and transduce the CNS while showing significantly reduced hepatic and vascular endothelial transduction.

rAAVrh.8, also isolated from rhesus macaques, shows a global transduction of glial and neuronal cell types in regions of clinical importance following peripheral administration and also displays reduced peripheral tissue tropism compared to other vectors.

AAV-BR1 is an AAV2 variant displaying the NRGTEWD (SEQ ID NO: 17) epitope that was isolated during in vivo screening of a random AAV display peptide library. It shows high specificity accompanied by high transgene expression in the brain with minimal off-target affinity (including for the liver) (Körbelin et al., EMBO Mol Med. 2016; 8 (6): 609).

AAV-PHP.S (Addgene, Watertown, MA) is a variant of AAV9 generated with the CREATE method that encodes the 7-mer sequence QAVRTSL (SEQ ID NO: 18), transduces neurons in the enteric nervous system, and strongly transduces peripheral sensory afferents entering the spinal cord and brain stem.

AAV-PHP.B (Addgene, Watertown, MA) is a variant of AAV9 generated with the CREATE method that encodes the 7-mer sequence TLAVPFK (SEQ ID NO: 19). It transfers genes throughout the CNS with higher efficiency than AAV9 and transduces the majority of astrocytes and neurons across multiple CNS regions.

AAV-PPS, an AAV2 variant created by insertion of the DSPAHPS (SEQ ID NO: 20) epitope into the capsid of AAV2, shows a dramatically improved brain tropism relative to AAV2.

For additional information regarding capsids that cross the blood brain barrier, see Chan et al., Nat. Neurosci. 2017 August: 20 (8): 1172-1179.

In particular embodiments, a capsid that results in transduction of targeted cell types in a primate following administration (e.g., i.v. administration) is chosen. In particular embodiments, a capsid that results in widespread transduction of tissue and cell types impacted by the loss of Scn1a following administration is chosen. In particular embodiments, targeted cell types are GABAergic neurons. In particular embodiments, targeted cell types are GABAergic neurons in the forebrain.

When an artificial expression construct is expressed in a targeted cell type, it is expressed in at least an intended cell type.

When an artificial expression construct is selectively expressed in selected cells, it leads to expression of the administered artificial expression construct in the intended cell type with limited to no expression in other cell types, as explained in additional detail below. In particular embodiments, limited expression in other cell types is less than 50% expression in a reference cell type as compared to a targeted cell type; less than 40% expression in a reference cell type as compared to a targeted cell type; less than 30% expression in a reference cell type as compared to a targeted cell type; less than 20% expression in a reference cell type as compared to a targeted cell type; or less than 10% expression in a reference cell type as compared to a targeted cell type. In particular embodiments, a reference cell type refers to non-targeted cells. The non-targeted cells can be within the same anatomical structure as the targeted cells and/or can project to a common anatomical area. In particular embodiments, a reference cell type is within an anatomical structure that is adjacent to an anatomical structure that includes the targeted cell type. In particular embodiments, a reference cell type is a non-targeted cell with a different RNA expression profile than the targeted cells.

In particular embodiments, the product of the sequence (e.g., coding sequence or non-coding sequence) may be expressed at low levels in non-selected cell types, for example at less than 1% or 1%, 2%, 3%, 5%, 10%, 15% or 20% of the levels at which the product is expressed in selected cells.

In particular embodiments, targeted cell types (e.g., neuronal, and/or non-neuronal) can be identified based on transcriptional profiles, such as those described in Tasic et al., Nature 563, 72-78 (2018) and Hodge et al., Nature 573, 61-68 (2019). For reference, the following description of cell types and distinguishing features is also provided:

Neocortical GABAergic neuron Subclasses:
All: Express GABA synthesis genes Gad1/GAD1 and Gad2/GAD2.
Lamp5, Sncg, Serpinf1, and Vip GABAergic neurons: Developmentally derived from neuronal progenitors from the caudal ganglionic eminence (CGE) or preoptic area (POA).
Sst and Pvalb GABAergic neurons: Developmentally derived from neuronal progenitors in the medial ganglionic eminence (MGE).
Lamp5 GABAergic neurons: Found in many neocortical layers, especially upper (L1-L2/3), and have mainly neurogliaform and single bouquet morphology.
Lamp5_Lhx6 GABAergic neurons: A subset of Lamp5 GABAergic neurons that co-express Lamp5 and Lhx6.
Sncg GABAergic neurons: Found in many neocortical layers, and have molecular overlaps with Lamp5 and Vip cells, but inconsistent expression of Lamp5 or vasoactive intestinal peptide (Vip), with more consistent expression of Sncg.
Serpinf1 GABAergic neurons: Found in many neocortical layers, and have molecular overlaps with Sncg and Vip cells, but inconsistent expression of Sncg or Vip, with more consistent expression of Serpinf1.
Vip GABAergic neurons: Found in many neocortical layers, but especially frequent in upper layers (L1-L4), and highly express the neurotransmitter Vip.
Sst GABAergic neurons: Found in many neocortical layers, but especially frequent in lower layers (L5-L6). They highly express the neurotransmitter somatostatin (Sst), and frequently block dendritic inputs to postsynaptic neurons. Included in this subclass are sleep-active Sst Chod/neurons (which also express Nos1 and Tacr1) that are highly distinct from other Sst neurons but express some shared marker genes including Sst. In human, SST gene expression is often detected in layer 1 LAMP5+ GABAergic neuron subtypes.
Pvalb GABAergic neurons: Found in many neocortical layers, but especially frequent in lower layers (L5-L6). They highly express the calcium-binding protein parvalbumin (Pvalb), express neuropeptide Tac1, and frequently dampen the output of postsynaptic neurons. Most fast-spiking GABAergic neurons express Pvalb strongly. Included in this subclass are chandelier cells, which have distinct, chandelier-like morphology and express the markers Cpne5 and Vipr2 in mouse, and NOG and UNC5B in human.
Meis2: A distinct subclass defined by a single type, only neocortical GABAergic neuron type that expresses Meis2 gene, and does not express some other genes that are expressed by all other neocortical GABAergic neuron types (for example, Thyl and Scn2b). This type is found in L6b and subcortical white matter.

Thalamus GABAergic neuron classes and subclasses:
All: Express GABA synthesis genes Gad1/GAD1 and/or Gad2/GAD2.
Thalamic reticular nucleus (TRN) neurons: Express GABA synthesis genes Gad1/GAD1 and Pvalb/PVALB.
(ii) Compositions for Administration. ASO, artificial expression constructs and/or vectors of the present disclosure (referred to herein as physiologically active components) can be formulated with a carrier that is suitable for administration to a cell, tissue slice, animal (e.g., mouse, non-human primate), or human. Physiologically active components within compositions described herein can be prepared in neutral forms, as freebases, or as pharmaceutically-acceptable salts.

Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Carriers of physiologically active components can include solvents, dispersion media, vehicles, coatings, diluents, isotonic and absorption delaying agents, buffers, solutions, suspensions, colloids, and the like. The use of such carriers for physiologically active components is well known in the art. Except insofar as any conventional media or agent is incompatible with the physiologically active components, it can be used with compositions as described herein.

The phrase “pharmaceutically-acceptable carriers” refer to carriers that do not produce an allergic or similar untoward reaction when administered to a human.

In particular embodiments, compositions can be formulated for intravenous, intraparenchymal, intraocular, intravitreal, parenteral, subcutaneous, intracerebroventricular, intramuscular, intrathecal, intraspinal, intraperitoneal, oral or nasal inhalation, or by direct injection in or application to one or more cells, tissues, or organs.

Compositions may include liposomes, lipids, lipid complexes, microspheres, microparticles, nanospheres, and/or nanoparticles.

The formation and use of liposomes is generally known to those of skill in the art. Liposomes have been developed with improved serum stability and circulation half-times (see, for instance, U.S. Pat. No. 5,741,516). Further, various methods of liposome and liposome like preparations as potential drug carriers have been described (see, for instance U.S. Pat. Nos. 5,567,434; 5,552,157; 5,565,213; 5,738,868; and 5,795,587).

The disclosure also provides for pharmaceutically acceptable nanocapsule formulations of the physiologically active components. Nanocapsules can generally entrap compounds in a stable and reproducible way (Quintanar-Guerrero et al., Drug Dev Ind Pharm 24 (12): 1113-1128, 1998; Quintanar-Guerrero et al., Pharm Res. 15 (7): 1056-1062, 1998; Quintanar-Guerrero et al., J. Microencapsul. 15 (1): 107-119, 1998; Douglas et al., Crit Rev Ther Drug Carrier Syst 3 (3): 233-261, 1987). To avoid side effects due to intracellular polymeric overloading, such ultrafine particles can be designed using polymers able to be degraded in vivo. Biodegradable polyalkyl-cyanoacrylate nanoparticles that meet these requirements are contemplated for use in the present disclosure. Such particles can be easily made, as described in Couvreur et al., J Pharm Sci 69 (2): 199-202, 1980; Couvreur et al., Crit Rev Ther Drug Carrier Syst. 5 (1) 1-20, 1988; zur Muhlen et al., Eur J Pharm Biopharm, 45 (2): 149-155, 1998; Zambaux et al., J Control Release 50 (1-3): 31-40, 1998; and U.S. Pat. No. 5,145,684.

Injectable compositions can include sterile aqueous solutions or dispersions and sterile powders for the extemporaneous preparation of sterile injectable solutions or dispersions (U.S. Pat. No. 5,466,468). For delivery via injection, the form is sterile and fluid to the extent that it can be delivered by syringe. In particular embodiments, it is stable under the conditions of manufacture and storage, and optionally contains one or more preservative compounds against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and/or antifungal agents, for example, parabens, chlorobutanol, phenol, sorbic acid, thimerosal, and the like. In various embodiments, the preparation will include an isotonic agent(s), for example, sugar(s) or sodium chloride. Prolonged absorption of the injectable compositions can be accomplished by including in the compositions of agents that delay absorption, for example, aluminum monostearate and gelatin. Injectable compositions can be suitably buffered, if necessary, and the liquid diluent first rendered isotonic with sufficient saline or glucose.

Dispersions may also be prepared in glycerol, liquid polyethylene glycols, and mixtures thereof and in oils. As indicated, under ordinary conditions of storage and use, these preparations can contain a preservative to prevent the growth of microorganisms.

Sterile compositions can be prepared by incorporating the physiologically active component in an appropriate amount of a solvent with other optional ingredients (e.g., as enumerated above), followed by filtered sterilization. Generally, dispersions are prepared by incorporating the various sterilized physiologically active components into a sterile vehicle that contains the basic dispersion medium and the required other ingredients (e.g., from those enumerated above). In the case of sterile powders for the preparation of sterile injectable solutions, preferred methods of preparation can be vacuum-drying and freeze-drying techniques which yield a powder of the physiologically active components plus any additional desired ingredient from a previously sterile-filtered solution thereof.

Oral compositions may be in liquid form, for example, as solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). Tablets may be coated by methods well-known in the art.

Inhalable compositions can be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Compositions can also include microchip devices (U.S. Pat. No. 5,797,898), ophthalmic formulations (Bourlais et al., Prog Retin Eye Res, 17 (1): 33-58, 1998), transdermal matrices (U.S. Pat. Nos. 5,770,219 and 5,783,208) and feedback-controlled delivery (U.S. Pat. No. 5,697,899).

Supplementary active ingredients can also be incorporated into the compositions.

Typically, compositions can include at least 0.1% of the physiologically active components or more, although the percentage of the physiologically active components may, of course, be varied and may conveniently be between 1 or 2% and 70% or 80% or more or 0.5-99% of the weight or volume of the total composition. Naturally, the amount of physiologically active components in each physiologically-useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of compositions and dosages may be desirable.

In particular embodiments, for administration to humans, compositions should meet sterility, pyrogenicity, and the general safety and purity standards as required by United States Food and Drug Administration (FDA) or other applicable regulatory agencies in other countries.
(iii) Cell Lines Including Artificial Expression Constructs. The present disclosure includes cells including an ASO, artificial expression construct, and/or vector described herein. A cell that has been transformed with an ASO, artificial expression construct, and/or vector can be used for many purposes, including in neuroanatomical studies, assessments of functioning and/or non-functioning proteins, and drug screens.

A variety of host cell lines can be used, but in particular embodiments, the cell is a mammalian cell. In particular embodiments, the cell line is a human, primate, or murine cell. Cell lines which can be utilized for transgenesis in the present disclosure also include primary cell lines derived from living tissue such as rat or mouse brains and organotypic cell cultures, including brain slices from animals such as rats, mice, non-human primates, or human neurosurgical tissue. The PC12 cell line (available from the American Type Culture Collection, ATCC, Manassas, VA) has been shown to express a number of neuronal marker proteins in response to Neuronal Growth Factor (NGF). The PC12 cell line is considered to be a neuronal cell line and is applicable for use with this disclosure. JAR cells (available from ATCC) are a platelet derived cell-line that express some neuronal genes, such as the serotonin transporter gene, and may be used with embodiments described herein.

WO 91/13150 describes a variety of cell lines, including neuronal cell lines, and methods of producing them. Similarly, WO 97/39117 describes a neuronal cell line and methods of producing such cell lines. The neuronal cell lines disclosed in these patent applications are applicable for use in the present disclosure.

In particular embodiments, “neuronal” describes something that is of, related to, or includes, neuronal cells. Neuronal cells are defined by the presence of an axon and dendrites. The term “neuronal-specific” refers to something that is found, or an activity that occurs, in neuronal cells or cells derived from neuronal cells, but is not found in or occur in, or is not found substantially in or occur substantially in, non-neuronal cells or cells not derived from neuronal cells, for example glial cells such as astrocytes or oligodendrocytes.

In particular embodiments, non-neuronal cell lines may be used, including mouse embryonic stem cells. Cultured mouse embryonic stem cells can be used to analyze expression of genetic constructs using transient transfection with plasmid constructs. Mouse embryonic stem cells are pluripotent and undifferentiated. These cells can be maintained in this undifferentiated state by Leukemia Inhibitory Factor (LIF). Withdrawal of LIF induces differentiation of the embryonic stem cells. In culture, the stem cells form a variety of differentiated cell types. Differentiation is caused by the expression of tissue specific transcription factors, allowing the function of an enhancer to be evaluated. (See for example Fiskerstrand et al., FEBS Lett 458:171-174, 1999).

Methods to differentiate stem cells into neuronal cells include replacing a stem cell culture media with a media including basic fibroblast growth factor (bFGF) heparin, an N2 supplement (e.g., transferrin, insulin, progesterone, putrescine, and selenite), laminin and polyornithine. A process to produce myelinating oligodendrocytes from stem cells is described in Hu, et al., 2009, Nat. Protoc. 4:1614-22. Bibel, et al., 2007, Nat. Protoc. 2:1034-43 describes a protocol to produce glutamatergic neurons from stem cells while Chatzi, et al., 2009, Exp. Neurol. 217:407-16 describes a procedure to produce GABAergic neurons. This procedure includes exposing stem cells to all-trans-RA for three days. After subsequent culture in serum-free neuronal induction medium including Neurobasal medium supplemented with B27, bFGF and EGF, 95% GABA neurons develop.

US 2012/0329714 describes use of prolactin to increase neural stem cell numbers while US 2012/0308530 describes a culture surface with amino groups that promotes neuronal differentiation into neurons, astrocytes and oligodendrocytes. Thus, the fate of neural stem cells can be controlled by a variety of extracellular factors. Commonly used factors include brain derived growth factor (BDNF; Shetty and Turner, 1998, J. Neurobiol. 35:395-425); fibroblast growth factor (bFGF; U.S. Pat. No. 5,766,948; FGF-1, FGF-2); Neurotrophin-3 (NT-3) and Neurotrophin-4 (NT-4); Caldwell, et al., 2001, Nat. Biotechnol. 1; 19:475-9); ciliary neurotrophic factor (CNTF); BMP-2 (U.S. Pat. Nos. 5,948,428 and 6,001,654); isobutyl 3-methylxanthine; leukemia inhibitory growth factor (LIF; U.S. Pat. No. 6,103,530); somatostatin; amphiregulin; neurotrophins (e.g., cyclic adenosine monophosphate; epidermal growth factor (EGF); dexamethasone (glucocorticoid hormone); forskolin; GDNF family receptor ligands; potassium; retinoic acid (U.S. Pat. No. 6,395,546); tetanus toxin; and transforming growth factor-α and TGF-β (U.S. Pat. Nos. 5,851,832 and 5,753,506).

Transgenic animals are described below. Cell lines may also be derived from such transgenic animals. For example, primary tissue culture from transgenic mice (e.g., also as described below) can provide cell lines with the artificial expression construct already integrated into the genome (for an example see Mackenzie & Quinn, Proc Natl Acad Sci USA 96:15251-15255, 1999).
(iv) Transgenic Animals. Another aspect of the disclosure includes transgenic animals, the genome of which contains an ASO, an artificial expression construct and/or vector disclosed herein. In particular embodiments, the genome of a transgenic animal includes CN4706, CN4992, and/or CN4993. In particular embodiments, when a non-integrating vector is utilized, a transgenic animal includes an ASO, artificial expression construct and/or vector disclosed herein within one or more of its cells.

Detailed methods for producing transgenic animals are described in U.S. Pat. No. 4,736,866. Transgenic animals may be of any nonhuman species, but preferably include nonhuman primates (NHPs), sheep, horses, cattle, pigs, goats, dogs, cats, rabbits, chickens, and rodents such as guinea pigs, hamsters, gerbils, rats, mice, and ferrets.

In particular embodiments, construction of a transgenic animal results in an organism that has an engineered construct present in all cells in the same genomic integration site. Thus, cell lines derived from such transgenic animals will be consistent in as much as the engineered construct will be in the same genomic integration site in all cells and hence will suffer the same position effect variegation. In contrast, introducing genes into cell lines or primary cell cultures can give rise to expression of the construct. A disadvantage of this approach is that the expression of the introduced DNA may be affected by the specific genetic background of the host animal.

As indicated above in relation to cell lines, the artificial expression constructs of this disclosure can be used to genetically modify mouse embryonic stem (ES) cells using techniques known in the art. Typically, the artificial expression construct is introduced into cultured murine embryonic stem cells. Transformed ES cells are then injected into a blastocyst from a host mother and the host embryo re-implanted into the mother. This results in a chimeric mouse whose tissues are composed of cells derived from both the embryonic stem cells present in the cultured cell line and the embryonic stem cells present in the host embryo. Usually, the mice from which the cultured ES cells used for transgenesis are derived are chosen to have a different coat color from the host mouse into whose embryos the transformed cells are to be injected. Chimeric mice will then have a variegated coat color. As long as the germ-line tissue is derived, at least in part, from the genetically modified cells, then the chimeric mice crossed with an appropriate strain can produce offspring that will carry the transgene.

In addition to the methods of delivery described above, the following techniques are also contemplated as alternative methods of delivering ASO, artificial expression constructs, and/or vectors to target cells or targeted tissues and organs of an animal, and in particular, to cells, organs, or tissues of a vertebrate mammal: sonophoresis (e.g., ultrasound, as described in U.S. Pat. No. 5,656,016); intraosseous injection (U.S. Pat. No. 5,779,708); microchip devices (U.S. Pat. No. 5,797,898); ophthalmic formulations (Bourlais et al., Prog Retin Eye Res, 17 (1): 33-58, 1998); transdermal matrices (U.S. Pat. Nos. 5,770,219 and 5,783,208); feedback-controlled delivery (U.S. Pat. No. 5,697,899), and any other delivery method available and/or described elsewhere in the disclosure.
(v) Methods of Use. In particular embodiments, a composition including a physiologically active component described herein is administered to a subject to result in a physiological effect.

In particular embodiments, the disclosure includes the use of the physiologically active components described herein to modulate protein expression. Thus, there are provided herein methods of use of the disclosed physiologically active components in the research, study, and development of medicaments for preventing, treating or ameliorating the symptoms of a disease, dysfunction, or disorder.

Particular embodiments include methods of administering to a subject an artificial expression construct that includes an miR binding site between a U7 promoter and an ASO as described herein to drive expression of an ASO in GABAergic neurons. The subject can be an isolated cell, a network of cells, a tissue slice, an experimental animal, a veterinary animal, or a human.

As is well known in the medical arts, dosages for any one subject depends upon many factors, including the subject's size, surface area, age, the particular compound to be administered, sex, time and route of administration, general health, and other drugs being administered concurrently. Dosages for the compounds of the disclosure will vary, but, in particular embodiments, a dose could be from 10⁵ to 10¹⁰⁰ copies of a physiologically active component of the disclosure. In particular embodiments, a patient receiving intravenous, intraparenchymal, intraspinal, retro-orbital, or intrathecal administration can be infused with from 10⁶ to 10²² copies of the physiologically active component.

Therapeutically effective amounts include those that provide effective amounts prophylactic treatments, and/or therapeutic treatments.

An “effective amount” is the amount of a composition necessary to result in a desired physiological change in the subject. Effective amounts are often administered for research purposes. Effective amounts disclosed herein can cause a statistically-significant effect in an animal model, human study, in vivo, or in vitro assay.

A “prophylactic treatment” includes a treatment administered to a subject who does not display signs or symptoms of a disorder or displays only early signs or symptoms of a disorder such that treatment is administered for the purpose of diminishing or decreasing the risk of developing the disorder further. Thus, a prophylactic treatment functions as a preventative treatment against a SCN1A-related disorder. In particular embodiments, prophylactic treatments reduce, delay, or prevent an SCN1A-related disorder or symptom from occurring.

A “therapeutic treatment” includes a treatment administered to a subject who displays symptoms or signs of a disorder and is administered to the subject for the purpose of diminishing or eliminating those signs or symptoms of the disorder. The therapeutic treatment can reduce, control, or eliminate the presence or activity of the disorder, the cause of the disorder, and/or reduce control or eliminate side effects of the disorder. In particular embodiments, the disorder is a disorder associated with Nav1.1 sodium channel dysfunction.

Function as an effective amount, prophylactic treatment, or therapeutic treatment are not mutually exclusive, and in particular embodiments, administered dosages may accomplish more than one treatment type.

In particular embodiments, the disclosure includes the use of the ASO of SEQ ID NOs: 1-12 or 30. In particular embodiments, the disclosure includes the use of the artificial expression constructs and/or vectors described herein to express an SCN1A ASO.

Thus, provided herein are methods of use of the disclosed physiologically active components in the research, study, and potential development of medicaments for preventing, treating or ameliorating the symptoms of a disease, dysfunction, or disorder related to sodium voltage-gated channel disfunction.

In particular embodiments, rescuing voltage-gated sodium channel function includes converting a subject's cells lacking a sufficient quantity and/or activity of Nav1.1 sodium channels, into cells that express a sufficient quantity of exogenous voltage-gated sodium channels and activity, in order to recover neuronal function and to reduce or prevent symptoms of voltage-gated sodium channel disfunction. Symptoms of voltage-gated sodium channel disfunction can include seizures, behavioral alterations, sleep disorders, cognitive impairment, walking difficulties, body temperature regulation problems, epileptiform circuit activity, trouble speaking, or delayed language development.

In particular embodiments, rescued voltage-gated sodium channel function is evidenced by one or more of an increase in sodium channel current in and/or the increased excitability of a targeted neuron genetically-modified by the physiologically active component. In particular embodiments, rescued voltage-gated sodium channel function is evidenced by one or more of an increase in sodium channel conductance in and/or the sodium channel influx in response to voltage depolarization of a targeted cell type genetically-modified by the physiologically active component. An increase can be at least a 10% increase, at least a 20% increase, at least a 30% increase, at least a 40% increase, at least a 50% increase, at least a 60% increase, at least a 70% increase, at least an 80% increase or at least a 90% increase. The output of GABAergic neurons can be measured using an electrophysiological method, such as a multi-electrode array or a patch-clamp.

In particular embodiments, rescued voltage-gated sodium channel function is evidenced by increased sodium current-dependent fast spiking in forebrain interneurons, for example, using a mouse model. In particular embodiments, rescued voltage-gated sodium channel function is evidenced by delayed or prevented temperature-induced seizing in a mouse model as described herein.

In particular embodiments, the targeted cell type is a GABAergic neuron. In particular embodiments, the GABAergic neuron is a pan-GABAergic neuron, a forebrain GABAergic neuron, a hippocampal GABAergic neuron, or a cortical GABAergic neuron.

Particular embodiments include identifying a subject with an SCN1A-related seizure disorder in targeted cell types (e.g., forebrain GABAergic neurons). Such subjects can be identified based on a diagnosis of a disorder associated with an SCN1A-related disorder.

In particular embodiments, physiologically active components are administered to a mouse model. In particular embodiments the mouse model includes an SCN1A^{R613X} mutant mouse. SCN1A^{R613X} mutant mice carry an A to T point mutation in nucleiotide 1837 which converts an arginine (R) 613 to a stop (X) codon. SCN1A^{R613X} mutant mice also have a silent C to T mutation at position 1833. The premature stop codon results in reduced SCN1A mRNA and Nav1.1 protein expression levels.

80% of patients with Dravet syndrome test positive for an SCN1A gene mutation, but the absence of an SCN1A mutation does not exclude a Dravet syndrome diagnosis. Dravet syndrome is associated with mutations in SCN1A (such as partial or total deletion mutations, truncating mutations and/or missense mutations e.g., in the voltage or pore regions S4 to S6), SCN1B (encoding the sodium channel (1 subunit), SCN2A, SCN3A, SCN9A, GABRG2 (encoding the γ2 subunit of GABA receptor), GABRD (encoding the delta subunit of GABA receptor) and/or PCDH19 genes.

The methods described herein may be particularly useful for treating children and infants, and for treating disorders that onset during infancy or childhood. In particular embodiments, the patient of the disclosed method is a newborn, a baby, a toddler, a preschooler, a school-age child, a tween, or a teenager. In particular embodiments, the patient is 18 years old or younger, 12 years old or younger, 10 years old or younger, 8 years old or younger, 6 years old or younger, 4 years old or younger, 2 years old or younger, 1 year old or younger. In particular embodiments, the patient is an adult that is over eighteen years old.

In particular embodiments, the methods reduce or prevent seizures, or symptoms thereof in a patient in need thereof. In particular embodiments, the methods provided may reduce or prevent one or more different types of seizures. Ideally, the methods of the disclosure result in a total prevention of seizures. However, the disclosure also encompasses methods in which the instances of seizures are decreased by at least 10%, at least 20%, at least 30%, at least 40%, at least 50%, at least 60%, at least 70%, at least 80% or at least 90%.

Generally, a seizure can include convulsions, repetitive movements, unusual sensations, and combinations thereof. Seizures can be categorized as focal seizures (also referred to as partial seizures) and generalized seizures. Focal seizures affect only one side of the brain, while generalized seizures affect both sides of the brain. Specific types of focal seizures include simple focal seizures, complex focal seizures, and secondarily generalized seizures. Simple focal seizures can be restricted or focused on a particular lobe (e.g., temporal lobe, frontal lobe, parietal lobe, or occipital lobe). Complex focal seizures generally affect a larger part of one hemisphere than simple focal seizures, but commonly originate in the temporal lobe or the frontal lobe. When a focal seizure spreads from one side (hemisphere) to both sides of the brain, the seizure is referred to as a secondarily generalized seizure. Specific types of generalized seizures include absences (also referred to as petit mal seizures), tonic seizures, atonic seizures, myoclonic seizures, tonic clonic seizures (also referred to as grand mal seizures), and clonic seizures.

In particular embodiments, methods described herein may reduce the frequency of seizures, reduce the severity of seizures, change the type of seizures (e.g., from a more severe type to a less severe type), or a combination thereof in a patient after treatment compared to the absence of treatment (e.g., before treatment), or compared to treatment with an alternative conventional treatment.

Disorders that may be treated with the physiologically active components disclosed herein include SCN1A-related disorders. Exemplary SCN1A-related disorders include DS (also known as severe myoclonic epilepsy of infancy or SMEI); SMEB; FS; GEFS+; epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; SUDEP; early infantile SCN1A encephalopathy; EIEE; sick sinus syndrome 1; autism; hemiplegic migraine; arthrogryposis multiplex congenita (AMC); myoclonic-atonic epilepsy (MAE); epilepsy of infancy with migrating focal seizures (EIMFS); Rett syndrome; nonsyndromic epileptic encephalopathy (NEE); or intractable childhood epilepsy with generalized tonic-clonic seizures (ICE-GTC).

In some instances, GEFS+ is epilepsy, generalized, with febrile seizures plus, type 2.

In some instances, the FS is FS, familial, 3A.

In some instances, SMEB is SMEB without generalized spike wave (SMEB-SW), SMEB without myoclonic seizures (SMEB-M), SMEB lacking more than one feature of SMEI (SMEB-O), or intractable childhood epilepsy with generalized tonic-clonic seizures (ICEGTC).

Administration of compositions disclosed herein can be by any appropriate route. For example, in particular embodiments, administration may include administration to a cell or tissue slice for research purposes related to Nav1.1 sodium channel dysfunction. In particular embodiments, administration to a cell or tissue can be done by pipette or injection.

In particular embodiments, administration is to a subject and can be intravenous, retro-orbital, intraocular, intravitreal, parenteral, subcutaneous, intracerebroventricular, intramuscular, intraparenchymal, intrathecal, intraspinal, intraperitoneal, oral, nasal, or direct to a targeted site administration. Delivery can be accomplished by a needle or a cannula or by any other technique of expelling fluidic materials.

The methods of administration may also include those modalities as described in U.S. Pat. Nos. 5,543,158; 5,641,515 and 5,399,363.

The amount of physiologically active components and time of administration of such compositions will be within the purview of the skilled artisan having benefit of the present teachings. It is likely, however, that the administration of the disclosed compositions may be achieved by a single administration, such as for example, a single injection of sufficient numbers of infectious particles to provide an effect in the subject. Alternatively, in some circumstances, it may be desirable to provide multiple, or successive administrations of the physiologically active components, either over a relatively short, or a relatively prolonged period of time, as may be determined by the individual overseeing the administration of such compositions. For example, the number of infectious particles administered to a mammal may be 107, 108, 109, 1010, 1011, 1012, 1013, or even higher, infectious particles/ml given either as a single dose or divided into two or more administrations as may be required to achieve an intended effect. In fact, in certain embodiments, it may be desirable to administer two or more different expression constructs in combination to achieve a desired effect.

In particular embodiments, treatments for Nav1.1 sodium channel disorders can be combined with another treatment. For example, common conventional therapies for seizures and epilepsy include antiepileptic drugs and non-antiepileptic drug treatments such as low carbohydrate diet (e.g., ketogenic diets, such as classical diet, medium chain triglyceride (MCT) diet, modified Atkins diet (MAD), and low glycemic index treatment (LGIT)), intravenous immunoglobulin, steroids, elimination diet, vagus nerve stimulation, corticetomy, and multiple subpial transections.

Common antiepileptic and anticonvulsive active compounds that may be used in combination with compositions described herein include acetazolamide, cannabidiol, carbamazepine, clobazam, clonazepam, eslicarbazepine acetate, ethosuximide, gabapentin, lacosamide, lamotrigine, levetiracetam, nitrazepam, oxcarbazepine, perampanel, piracetam, phenobarbital, phenytoin, pregabalin, primidone, retigabine, rufinamide, sodium valproate, stiripentol, tiagabine, topiramate, vigabatrin, and zonisamide.
(vi) Kits and Commercial Packages. Kits and commercial packages contain a physiologically active component described herein. The physiologically active component can be isolated or provided within a composition. In particular embodiments, the components of a physiologically active component can be isolated from each other. In particular embodiments, an ASO or artificial expression construct can be within a vector, within a viral vector, within a cell, within a tissue slice or sample, and/or within a transgenic animal. In particular embodiments, an animal is transgenic following administration of a composition including the physiologically active component. In particular embodiments, a transgenic animal includes a genetic modification that renders the animal appropriate for use in an animal model of Dravet syndrome. For example, the transgenic animal such as a mouse can be Scn1a+/−.

Such kits may further include one or more reagents, restriction enzymes, peptides, therapeutics, pharmaceutical compounds, or means for delivery of the compositions such as syringes, injectables, and the like.

Embodiments of a kit or commercial package will also contain instructions regarding use of the included components, for example, in basic research, electrophysiological research, neuroanatomical research, and/or the research and/or treatment of a disorder, disease or condition (e.g., Nav1.1 sodium channel dysfunction, such as epilepsy and/or Dravet syndrome).

The Exemplary Embodiments below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.
(vii) Exemplary Embodiments.
1. An antisense oligonucleotide (ASO) encoded by a sequence as set forth in any one of SEQ ID NOs: 1-12 or 30 or a sequence having at least 98% sequence identity to a sequence as set forth in any one of SEQ ID NOs: 1-12 or 30.
2. The ASO of embodiment 1, wherein the ASO is encoded by the sequence as set forth in SEQ ID NO: 1.
3. The ASO of embodiments 1, wherein the ASO is encoded by the sequence as set forth in SEQ ID NO: 2.
4. The ASO of embodiment 1, wherein the ASO is encoded by the sequence as set forth in SEQ ID NO: 3.
5. The ASO of embodiment 1, wherein the ASO is encoded by the sequence as set forth in SEQ ID NO: 4.
6. The ASO of embodiment 1, wherein the ASO is encoded by the sequence as set forth in SEQ ID NO: 5.
7. The ASO of embodiment 1, wherein the ASO is encoded by the sequence as set forth in SEQ ID NO: 6.
8. The ASO of embodiment 1, wherein the ASO is encoded by the sequence as set forth in SEQ ID NO: 7.
9. The ASO of embodiment 1, wherein the ASO is encoded by the sequence as set forth in SEQ ID NO: 8.
10. The ASO of embodiment 1, wherein the ASO is encoded by the sequence as set forth in SEQ ID NO: 9.
11. The ASO of embodiment 1, wherein the ASO is encoded by the sequence as set forth in SEQ ID NO: 10.
12. The ASO of embodiment 1, wherein the ASO is encoded by the sequence as set forth in SEQ ID NO: 11.
13. The ASO of embodiment 1, wherein the ASO is encoded by the sequence as set forth in SEQ ID NO: 12.
14. The ASO of embodiment 1, wherein the ASO is encoded by the sequence as set forth in SEQ ID NO: 30.
15. The ASO of any of embodiments 1-14, wherein the ASO includes a 2′-O-methoxyethyl base.
16. The ASO of any of embodiments 1-15, wherein each nucleotide of the ASO includes a 2′-O-methoxyethyl base.
17. The ASO of any of embodiments 1-16, wherein the ASO includes a phosphorothioate internucleotide linkage.
18. The ASO of any of embodiments 1-17, wherein each nucleotide of the ASO includes a phosphorothioate internucleotide linkage.
19. An artificial expression construct including or encoding (i) an enhancer, (ii) a U7 promoter, (iii) an antisense oligonucleotide (ASO); (iv) an Sm binding domain, and (v) a 3′ box.
20. The artificial expression construct of embodiment 19, wherein the ASO is encoded by:
the sequence as set forth in SEQ ID NO: 1 or a sequence having at least 98% sequence identity to the sequence as set forth in SEQ ID NO:1,
the sequence as set forth in SEQ ID NO: 2 or a sequence having at least 98% sequence identity to the sequence as set forth in SEQ ID NO: 2,
the sequence as set forth in SEQ ID NO: 3 or a sequence having at least 98% sequence identity to the sequence as set forth in SEQ ID NO: 3,
the sequence as set forth in SEQ ID NO: 4 or a sequence having at least 98% sequence identity to the sequence as set forth in SEQ ID NO: 4,
the sequence as set forth in SEQ ID NO: 5 or a sequence having at least 98% sequence identity to the sequence as set forth in SEQ ID NO: 5,
the sequence as set forth in SEQ ID NO: 6 or a sequence having at least 98% sequence identity to the sequence as set forth in SEQ ID NO: 6,
the sequence as set forth in SEQ ID NO: 7 or a sequence having at least 98% sequence identity to the sequence as set forth in SEQ ID NO: 7,
the sequence as set forth in SEQ ID NO: 8 or a sequence having at least 98% sequence identity to the sequence as set forth in SEQ ID NO: 8,
the sequence as set forth in SEQ ID NO: 9 or a sequence having at least 98% sequence identity to the sequence as set forth in SEQ ID NO: 9,
the sequence as set forth in SEQ ID NO: 10 or a sequence having at least 98% sequence identity to the sequence as set forth in SEQ ID NO: 10,
the sequence as set forth in SEQ ID NO: 11 or a sequence having at least 98% sequence identity to the sequence as set forth in SEQ ID NO: 11,
the sequence as set forth in SEQ ID NO: 12 or a sequence having at least 98% sequence identity to the sequence as set forth in SEQ ID NO: 12, or
the sequence as set forth in SEQ ID NO: 30 or a sequence having at least 98% sequence identity to the sequence as set forth in SEQ ID NO: 30.
21. The artificial expression construct of embodiments 19 or 20, wherein the U7 promoter includes the sequence as set forth in SEQ ID NO: 23 or a sequence having at least 95% sequence identity to the sequence as set forth in SEQ ID NO: 23.
22. The artificial expression construct of any of embodiments 19-1, wherein the enhancer includes SEQ ID NO: 21 or SEQ ID NO: 22 or a sequence having at least 95% sequence identity to the sequence as set forth in SEQ ID NO: 21 or SEQ ID NO: 22.
23. The artificial expression construct of any of embodiments 19-22, wherein the Sm binding domain includes a consensus Sm sequence (SmOpt).
24 The artificial expression construct of any of embodiments 19-23, wherein the Sm binding domain is encoded by the sequence as set forth in SEQ ID NO: 31 or SEQ ID NO: 32 or a sequence having at least 95% sequence identity to the sequence as set forth in SEQ ID NO: 31 or SEQ ID NO: 32.
25. The artificial expression construct of any of embodiments 19-24, wherein the 3′ box is encoded by the sequence as set forth in SEQ ID NO: 33 or SEQ ID NO: 34 or a sequence having at least 95% sequence identity to the sequence as set forth in SEQ ID NO: 33 or SEQ ID NO: 34.
26. The artificial expression construct of any of embodiments 19-25, further encoding a microRNA (miR) binding site.
27. The artificial expression construct of embodiment 26, wherein the miR binding site is encoded by the sequence as set forth in SEQ ID NO: 24 or SEQ ID NO: 25 or a sequence having at least 98% sequence identity to the sequence as set forth in SEQ ID NO: 24 or SEQ ID NO: 25.
28. The artificial expression construct of embodiments 26 or 27, wherein the miR binding site is encoded by a sequence having at least 98% sequence identity to the sequence as set forth in SEQ ID NO: 24 and a sequence having at least 98% sequence identity to the sequence as set forth in SEQ ID NO: 25.
29. The artificial expression construct of any of embodiments 26-28, wherein the miR binding site is encoded by the sequence set forth in SEQ ID NO: 24 and SEQ ID NO: 25.
30. The artificial expression construct of any of embodiments 26-29, wherein the miR binding site is encoded by a sequence having at least 98% sequence identity to the sequence set forth in SEQ ID NOs: 26, 27, 28, 29, 43, or 44.
31. The artificial expression construct of any of embodiments 26-30, wherein the miR binding site is encoded by the sequence as set forth in SEQ ID NOs: 26, 27, 28, 29, 43, or 44.
32. The artificial expression construct of any of embodiments 26-31, wherein the sequence encoding the miR binding site is between the U7 promoter and the sequence encoding the ASO, between the sequence encoding the ASO and the sequence encoding the Sm binding domain, between the sequence encoding the Sm binding domain and the sequence encoding the 3′ box, or 3′ of the sequence encoding the 3′ box.
33. The artificial expression construct of any of embodiments 26-32, wherein the sequence encoding the miR binding site is between the U7 promoter and the sequence encoding the ASO.
34. The artificial expression construct of any of embodiments 19-33, wherein the artificial expression construct includes or encodes a set of features selected from:
[enhancer]-[U7promoter]-[miR binding site]-[ASO]-[smOPT]-[3′ box]; or
[enhancer]-[U7promoter]-[ASO]-[smOPT]-[3′ box].
35. The artificial expression construct of any of embodiments 19-34, wherein the artificial expression construct includes or encodes a set of features selected from:
[hl56i (core)]-[U7promoter]-[miR binding site]-[ASO]-[smOPT]-[3′ box];
[DLX2.0]-[U7promoter]-[miR binding site]-[ASO]-[smOPT]-[3′ box];
[hl56i (core)]-[U7promoter]-[ASO]-[smOPT]-[3′ box]; or
[DLX2.0]-[U7promoter]-[ASO]-[smOPT]-[3′ box].
36. The artificial expression construct of any of embodiments 19-35, wherein the artificial expression construct includes or encodes a set of features selected from:
[hl56i (core)]-[U7promoter]-[miR-128 binding site]-[miR-221 binding site]-[ASO]-[smOPT]-[3′ box];
[DLX2.0]-[U7promoter]-[miR-128 binding site]-[miR-221 binding site]-[ASO]-[smOPT]-[3′ box];
[hl56i (core)]-[U7promoter]-[ASO]-[smOPT]-[3′ box]; or
[DLX2.0]-[U7promoter]-[ASO]-[smOPT]-[3′ box].
37. The artificial expression construct of any of embodiments 19-36, wherein the artificial expression construct includes or encodes a set of features selected from:
[hl56i (core)]-[U7promoter]-[miR-128 binding site]-[miR-221 binding site]-[SEQ ID NO: 1]-[smOPT]-[3′ box];
[DLX2.0]-[U7promoter]-[miR-128 binding site]-[miR-221 binding site]-[SEQ ID NO: 1]-[smOPT]-[3′ box];
[hl56i (core)]-[U7promoter]-[SEQ ID NO: 1]-[smOPT]-[3′ box];
[DLX2.0]-[U7promoter]-[SEQ ID NO: 1]-[smOPT]-[3′ box];
[hl56i (core)]-[U7promoter]-[miR-128 binding site]-[miR-221 binding site]-[SEQ ID NO: 2]-[smOPT]-[3′ box];
[DLX2.0]-[U7promoter]-[miR-128 binding site]-[miR-221 binding site]-[SEQ ID NO: 2]-[smOPT]-[3′ box];
[hl56i (core)]-[U7promoter]-[SEQ ID NO: 2]-[smOPT]-[3′ box];
[DLX2.0]-[U7promoter]-[SEQ ID NO: 2]-[smOPT]-[3′ box];
[hl56i (core)]-[U7promoter]-[miR-128 binding site]-[miR-221 binding site]-[SEQ ID NO: 3]-[smOPT]-[3′ box];
[DLX2.0]-[U7promoter]-[miR-128 binding site]-[miR-221 binding site]-[SEQ ID NO: 3]-[smOPT]-[3′ box];
[hl56i (core)]-[U7promoter]-[SEQ ID NO: 3]-[smOPT]-[3′ box];
[DLX2.0]-[U7promoter]-[SEQ ID NO: 3]-[smOPT]-[3′ box];
[hl56i (core)]-[U7promoter]-[miR-128 binding site]-[miR-221 binding site]-[SEQ ID NO: 4]-[smOPT]-[3′ box];
[DLX2.0]-[U7promoter]-[miR-128 binding site]-[miR-221 binding site]-[SEQ ID NO: 4]-[smOPT]-[3′ box];
[hl56i (core)]-[U7promoter]-[SEQ ID NO: 4]-[smOPT]-[3′ box];
[DLX2.0]-[U7promoter]-[SEQ ID NO: 4]-[smOPT]-[3′ box];
[hl56i (core)]-[U7promoter]-[miR-128 binding site]-[miR-221 binding site]-[SEQ ID NO: 5]-[smOPT]-[3′ box];
[DLX2.0]-[U7promoter]-[miR-128 binding site]-[miR-221 binding site]-[SEQ ID NO: 5]-[smOPT]-[3′ box];
[hl56i (core)]-[U7promoter]-[SEQ ID NO: 5]-[smOPT]-[3′ box];
[DLX2.0]-[U7promoter]-[SEQ ID NO: 5]-[smOPT]-[3′ box];
[hl56i (core)]-[U7promoter]-[miR-128 binding site]-[miR-221 binding site]-[SEQ ID NO: 6]-[smOPT]-[3′ box];
[DLX2.0]-[U7promoter]-[miR-128 binding site]-[miR-221 binding site]-[SEQ ID NO: 6]-[smOPT]-[3′ box];
[hl56i (core)]-[U7promoter]-[SEQ ID NO: 6]-[smOPT]-[3′ box];
[DLX2.0]-[U7promoter]-[SEQ ID NO: 6]-[smOPT]-[3′ box];
[hl56i (core)]-[U7promoter]-[miR-128 binding site]-[miR-221 binding site]-[SEQ ID NO: 7]-[smOPT]-[3′ box];
[DLX2.0]-[U7promoter]-[miR-128 binding site]-[miR-221 binding site]-[SEQ ID NO: 7]-[smOPT]-[3′ box];
[hl56i (core)]-[U7promoter]-[SEQ ID NO: 7]-[smOPT]-[3′ box];
[DLX2.0]-[U7promoter]-[SEQ ID NO: 7]-[smOPT]-[3′ box];
[hl56i (core)]-[U7promoter]-[miR-128 binding site]-[miR-221 binding site]-[SEQ ID NO: 8]-[smOPT]-[3′ box];
[DLX2.0]-[U7promoter]-[miR-128 binding site]-[miR-221 binding site]-[SEQ ID NO: 8]-[smOPT]-[3′ box];
[hl56i (core)]-[U7promoter]-[SEQ ID NO: 8]-[smOPT]-[3′ box];
[DLX2.0]-[U7promoter]-[SEQ ID NO: 8]-[smOPT]-[3′ box];
[hl56i (core)]-[U7promoter]-[miR-128 binding site]-[miR-221 binding site]-[SEQ ID NO: 9]-[smOPT]-[3′ box];
[DLX2.0]-[U7promoter]-[miR-128 binding site]-[miR-221 binding site]-[SEQ ID NO: 9]-[smOPT]-[3′ box];
[hl56i (core)]-[U7promoter]-[SEQ ID NO: 9]-[smOPT]-[3′ box];
[DLX2.0]-[U7promoter]-[SEQ ID NO: 9]-[smOPT]-[3′ box];
[hl56i (core)]-[U7promoter]-[miR-128 binding site]-[miR-221 binding site]-[SEQ ID NO: 10]-[smOPT]-[3′ box];
[DLX2.0]-[U7promoter]-[miR-128 binding site]-[miR-221 binding site]-[SEQ ID NO: 10]-[smOPT]-[3′ box];
[hl56i (core)]-[U7promoter]-[SEQ ID NO: 10]-[smOPT]-[3′ box];
[DLX2.0]-[U7promoter]-[SEQ ID NO: 10]-[smOPT]-[3′ box];
[hl56i (core)]-[U7promoter]-[miR-128 binding site]-[miR-221 binding site]-[SEQ ID NO: 11]-[smOPT]-[3′ box];
[DLX2.0]-[U7promoter]-[miR-128 binding site]-[miR-221 binding site]-[SEQ ID NO: 11]-[smOPT]-[3′ box];
[hl56i (core)]-[U7promoter]-[SEQ ID NO: 11]-[smOPT]-[3′ box];
[DLX2.0]-[U7promoter]-[SEQ ID NO: 11]-[smOPT]-[3′ box];
[hl56i (core)]-[U7promoter]-[miR-128 binding site]-[miR-221 binding site]-[SEQ ID NO: 12]-[smOPT]-[3′ box];
[DLX2.0]-[U7promoter]-[miR-128 binding site]-[miR-221 binding site]-[SEQ ID NO: 12]-[smOPT]-[3′ box];
[hl56i (core)]-[U7promoter]-[SEQ ID NO: 12]-[smOPT]-[3′ box];
[DLX2.0]-[U7promoter]-[SEQ ID NO: 12]-[smOPT]-[3′ box];
[hl56i (core)]-[U7promoter]-[miR-128 binding site]-[miR-221 binding site]-[SEQ ID NO: 30]-[smOPT]-[3′ box];
[DLX2.0]-[U7promoter]-[miR-128 binding site]-[miR-221 binding site]-[SEQ ID NO: 30]-[smOPT]-[3′ box]. [hl56i (core)]-[U7promoter]-[SEQ ID NO: 30]-[smOPT]-[3′ box]; or
[DLX2.0]-[U7promoter]-[SEQ ID NO: 30]-[smOPT]-[3′ box].
38. The artificial expression construct of any of embodiments 19-37, wherein the artificial expression construct includes:
the sequence as set forth in SEQ ID NO: 35 or a sequence having at least 95% sequence identity to the sequence as set forth in SEQ ID NO: 35;
the sequence as set forth in SEQ ID NO: 36 or a sequence having at least 95% sequence identity to the sequence as set forth in SEQ ID NO: 36; or
the sequence as set forth in SEQ ID NO: 37 or a sequence having at least 95% sequence identity to the sequence as set forth in SEQ ID NO: 37.
39. The artificial expression construct of any of embodiments 19-38, wherein the artificial expression construct includes the sequence as set forth in SEQ ID NO: 35.
40. The artificial expression construct of any of embodiments 19-38, wherein the artificial expression construct includes the sequence as set forth in SEQ ID NO: 36.
41. The artificial expression construct of any of embodiments 19-38, wherein the artificial expression construct includes the sequence as set forth in SEQ ID NO: 37.
42. The artificial expression construct of any of embodiments 19-41, wherein the artificial expression construct is associated with a capsid that crosses a blood brain barrier.
43. The artificial expression construct of embodiment 42, wherein the capsid includes PHP.eB, AAV9, AAVrh. 10, AAV-BR1, AAV-PHP.S, AAV-PHP.B, or AAV-PPS.
44. A vector including an artificial expression construct of any of embodiments 19-43.
45. The vector of embodiment 44, wherein the vector includes a viral vector.
46. The vector of embodiment 45, wherein the viral vector includes a recombinant adeno-associated viral (AAV) vector.
47. An adeno-associated viral (AAV) vector including at least one artificial expression construct of any of embodiments 19-43.
48. A transgenic cell including an antisense oligonucleotide (ASO) of any of embodiments 1-18, an artificial expression construct of any of embodiments 19-43, and/or a vector of any of embodiments 44-46.
49. The transgenic cell of embodiment 48, wherein the transgenic cell is a GABAergic neuron.
50. The transgenic cell of embodiment 49, wherein the GABAergic neuron is a pan-GABAergic neuron, a forebrain GABAergic neuron, a hippocampal GABAergic neuron, or a cortical GABAergic neuron.
51. The transgenic cell of any of embodiments 48-50, wherein the transgenic cell is murine, human, or non-human primate.
52. A non-human transgenic animal including an antisense oligonucleotide (ASO) of any of embodiments 1-18, an artificial expression construct of any of embodiments 19-43, a vector of any of embodiments 44-46, and/or a transgenic cell of any of embodiments 48-51.
53. The non-human transgenic animal of embodiment 52, wherein the non-human transgenic animal is a mouse or a non-human primate.
54. An administrable composition including a pharmaceutically acceptable carrier and an antisense oligonucleotide (ASO) of any of embodiments 1-18, an artificial expression construct of any of embodiments 19-43, a vector of any of embodiments 44-46, and/or a transgenic cell of any of embodiments 48-51.
55. A kit including an antisense oligonucleotide (ASO) of any of embodiments 1-18, an artificial expression construct of any of embodiments 19-43, a vector of any of embodiments 44-46, a transgenic cell of any of embodiments 48-51, and/or an administrable composition of embodiment 54.
56. A method for providing an antisense oligonucleotide (ASO) within a targeted population of cells in vivo or in vitro, the method including administering the administrable composition of embodiment 54 in a sufficient dosage and for a sufficient time to a sample or subject including the targeted population of cells thereby providing the ASO within the population of cells.
57. The method of embodiment 56, wherein the ASO is 12-24 nucleotides in length.
58. The method of embodiments 56 or 57, wherein the ASO is 15-21 nucleotides in length.
59. The method of any of embodiments 56-58, wherein the ASO is 18 nucleotides in length.
60. The method of any of embodiments 56-59, wherein the ASO is encoded by at least one of SEQ ID NOs: 1-12 or 30 or a sequence having at least 98% sequence identity to the sequence as set forth in SEQ ID NOs: 1-12 or 30.
61. The method of any of embodiments 56-60, wherein the administering includes pipetting.
62. The method of embodiment 61, wherein the pipetting is to a brain slice.
63. The method of embodiment 62, wherein the brain slice includes a GABAergic neuron.
64. The method of embodiment 63, wherein the GABAergic neuron includes a pan-GABAergic neuron, a forebrain GABAergic neuron, a hippocampal GABAergic neuron, or a cortical GABAergic neuron.
65. The method of any of embodiments 62-64, wherein the brain slice is murine, human, or non-human primate.
66. The method of any of embodiments 56-65, wherein the administering includes administering to a living subject.
67. The method of embodiment 66, wherein the living subject is a human, non-human primate, or a mouse.
68. The method of embodiments 66 or 67, wherein the living subject has an SCN1A-related disorder.
69. The method of embodiment 68, wherein the SCN1A-related disorder includes Dravet Syndrome (DS); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); sick sinus syndrome 1; early infantile SCN1A encephalopathy; early infantile epileptic encephalopathy (EIEE); autism; hemiplegic migraine; arthrogryposis multiplex congenita (AMC); myoclonic-atonic epilepsy (MAE); epilepsy of infancy with migrating focal seizures (EIMFS); Rett syndrome; nonsyndromic epileptic encephalopathy (NEE); or intractable childhood epilepsy with generalized tonic-clonic seizures (ICE-GTC).
70. The method of any of embodiments 66-69, wherein the living subject is a pediatric patient.
71. The method of any of embodiments 66-70, wherein the living subject is less than 4 years old.
72. The method of any of embodiments 66-69, wherein the living subject is an adult.
73. The method of any of embodiments 66-69, wherein the living subject is a transgenic Scn1aR613X mutant mouse.
74. The method of any of embodiments 56-73, wherein the administering to a living subject is through injection.
75. The method of embodiment 74, wherein the injection includes intracerebroventricular (ICV) injection, intravenous injection, intraparenchymal injection into brain tissue, intra-cisterna magna (ICM) injection, or intrathecal injection.
76. The method of any of embodiments 56-75, wherein the composition is administered by intracerebroventricular (ICV) injection.
77. The method of any of embodiments 56-76, wherein the providing rescues voltage-gated sodium channel function in a cell in need thereof, with a proviso that the ASO does not include a sequence encoded by SEQ ID NO: 4.
(viii) Closing Paragraphs. The nucleic acid and amino acid sequences provided herein are shown using letter abbreviations for nucleotide bases and amino acid residues, as defined in 37 C.F.R. § 1.831-1.835 and set forth in WIPO Standard ST.26 (implemented on Jul. 1, 2022). Only one strand of each nucleic acid sequence is shown, but the complementary strand is understood as included in embodiments where it would be appropriate.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (11e), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, 11e, Phe, Met, and Trp; Group 11 (aliphatic): Gly, Ala, Val, Leu, and 11e; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157 (1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within +2 is preferred, those within +1 are particularly preferred, and those within +0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); 11e (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y., Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42 °C in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50 °C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37 °C in a solution including 6×SSPE (20×SSPE=3M NaCl; 0.2M NaH₂PO₄; 0.02M EDTA, pH 7.4), 0.5% SDS, 30% formamide, 100 μg/ml salmon sperm blocking DNA; followed by washes at 50 °C with 1×SSPE, 0.1% SDS. In addition, to achieve even lower stringency, washes performed following stringent hybridization can be done at higher salt concentrations (e.g., 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The term concatenate is broadly used to describe linking together into a chain or series. It is used to describe the linking together of nucleotide or amino acid sequences into a single nucleotide or amino acid sequence, respectively. The term “concatamerize” should be interpreted to recite: “concatenate.”

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in targeted expression in GABAergic neurons.

In particular embodiments, artificial means not naturally occurring.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. An artificial expression construct comprising or encoding a DLX2.0 enhancer, a U7 promoter, an miR-128 binding site, an miR-221 binding site, an ASO as set forth by SEQ ID NO: 30, a consensus Sm sequence (SmOpt), and a 3′ box.

2. The artificial expression construct of claim 1, comprising the sequence as set forth in SEQ ID NO: 35.

3. An antisense oligonucleotide (ASO) encoded by a sequence as set forth in any one of SEQ ID NOs: 1-12 or 30 or a sequence having at least 98% sequence identity to a sequence as set forth in any one of SEQ ID NOs: 1-12 or 30.

4. The ASO of claim 3, wherein the ASO comprises a 2′-O-methoxyethyl base.

5. The ASO of claim 3, wherein each nucleotide of the ASO comprises a 2′-O-methoxyethyl base.

6. The ASO of claim 3, wherein the ASO comprises a phosphorothioate internucleotide linkage.

7. The ASO of claim 3, wherein each nucleotide of the ASO comprises a phosphorothioate internucleotide linkage.

8. An artificial expression construct comprising or encoding (i) an enhancer, (ii) a U7 promoter, (iii) an antisense oligonucleotide (ASO); (iv) an Sm binding domain, and (v) a 3′ box.

9. The artificial expression construct of claim 8, wherein the ASO is encoded by:
the sequence as set forth in SEQ ID NO: 1 or a sequence having at least 98% sequence identity to the sequence as set forth in SEQ ID NO:1,
the sequence as set forth in SEQ ID NO: 2 or a sequence having at least 98% sequence identity to the sequence as set forth in SEQ ID NO: 2,
the sequence as set forth in SEQ ID NO: 3 or a sequence having at least 98% sequence identity to the sequence as set forth in SEQ ID NO: 3,
the sequence as set forth in SEQ ID NO: 4 or a sequence having at least 98% sequence identity to the sequence as set forth in SEQ ID NO: 4,
the sequence as set forth in SEQ ID NO: 5 or a sequence having at least 98% sequence identity to the sequence as set forth in SEQ ID NO: 5,
the sequence as set forth in SEQ ID NO: 6 or a sequence having at least 98% sequence identity to the sequence as set forth in SEQ ID NO: 6,
the sequence as set forth in SEQ ID NO: 7 or a sequence having at least 98% sequence identity to the sequence as set forth in SEQ ID NO: 7,
the sequence as set forth in SEQ ID NO: 8 or a sequence having at least 98% sequence identity to the sequence as set forth in SEQ ID NO: 8,
the sequence as set forth in SEQ ID NO: 9 or a sequence having at least 98% sequence identity to the sequence as set forth in SEQ ID NO: 9,
the sequence as set forth in SEQ ID NO: 10 or a sequence having at least 98% sequence identity to the sequence as set forth in SEQ ID NO: 10,
the sequence as set forth in SEQ ID NO: 11 or a sequence having at least 98% sequence identity to the sequence as set forth in SEQ ID NO: 11,
the sequence as set forth in SEQ ID NO: 12 or a sequence having at least 98% sequence identity to the sequence as set forth in SEQ ID NO: 12, or
the sequence as set forth in SEQ ID NO: 30 or a sequence having at least 98% sequence identity to the sequence as set forth in SEQ ID NO: 30.

10. The artificial expression construct of claim 8, wherein the U7 promoter comprises the sequence as set forth in SEQ ID NO: 23 or a sequence having at least 95% sequence identity to the sequence as set forth in SEQ ID NO: 23.

11. The artificial expression construct of claim 8, wherein the enhancer comprises SEQ ID NO: 21 or SEQ ID NO: 22 or a sequence having at least 95% sequence identity to the sequence as set forth in SEQ ID NO: 21 or SEQ ID NO: 22.

12. The artificial expression construct of claim 8, wherein the Sm binding domain comprises a consensus Sm sequence (SmOpt).

13. The artificial expression construct of claim 8, wherein the Sm binding domain is encoded by the sequence as set forth in SEQ ID NO: 31 or SEQ ID NO: 32 or a sequence having at least 95% sequence identity to the sequence as set forth in SEQ ID NO: 31 or SEQ ID NO: 32.

14. The artificial expression construct of claim 8, wherein the 3′ box is encoded by the sequence as set forth in SEQ ID NO: 33 or SEQ ID NO: 34 or a sequence having at least 95% sequence identity to the sequence as set forth in SEQ ID NO: 33 or SEQ ID NO: 34.

15. The artificial expression construct of claim 8, further encoding a microRNA (miR) binding site.

16. The artificial expression construct of claim 15, wherein the miR binding site is encoded by the sequence as set forth in SEQ ID NO: 24 or SEQ ID NO: 25 or a sequence having at least 98% sequence identity to the sequence as set forth in SEQ ID NO: 24 or SEQ ID NO: 25.

17. The artificial expression construct of claim 15, wherein the miR binding site is encoded by a sequence having at least 98% sequence identity to the sequence as set forth in SEQ ID NO: 24 and a sequence having at least 98% sequence identity to the sequence as set forth in SEQ ID NO: 25.

18. The artificial expression construct of claim 15, wherein the miR binding site is encoded by the sequence set forth in SEQ ID NO: 24 and SEQ ID NO: 25.

19. The artificial expression construct of claim 15, wherein the miR binding site is encoded by a sequence having at least 98% sequence identity to the sequence set forth in SEQ ID NOs: 26, 27, 28, 29, 43, or 44.

20. The artificial expression construct of claim 15, wherein the miR binding site is encoded by the sequence as set forth in SEQ ID NOs: 26, 27, 28, 29, 43, or 44.

21. The artificial expression construct of claim 15, wherein the sequence encoding the miR binding site is between the U7 promoter and the sequence encoding the ASO, between the sequence encoding the ASO and the sequence encoding the Sm binding domain, between the sequence encoding the Sm binding domain and the sequence encoding the 3′ box, or 3′ of the sequence encoding the 3′ box.

22. The artificial expression construct of claim 15, wherein the sequence encoding the miR binding site is between the U7 promoter and the sequence encoding the ASO.

23. The artificial expression construct of claim 8, wherein the artificial expression construct comprises or encodes a set of features selected from:
[enhancer]-[U7promoter]-[miR binding site]-[ASO]-[smOPT]-[3′ box]; or
[enhancer]-[U7promoter]-[ASO]-[smOPT]-[3′ box].

24. The artificial expression construct of claim 8, wherein the artificial expression construct comprises or encodes a set of features selected from:
[hl56i (core)]-[U7promoter]-[miR binding site]-[ASO]-[smOPT]-[3′ box];
[DLX2.0]-[U7promoter]-[miR binding site]-[ASO]-[smOPT]-[3′ box];
[hl56i (core)]-[U7promoter]-[ASO]-[smOPT]-[3′ box]; or
[DLX2.0]-[U7promoter]-[ASO]-[smOPT]-[3′ box].

25. The artificial expression construct of claim 8, wherein the artificial expression construct comprises or encodes a set of features selected from:
[hl56i (core)]-[U7promoter]-[miR-128 binding site]-[miR-221 binding site]-[ASO]-[smOPT]-[3′ box];
[DLX2.0]-[U7promoter]-[miR-128 binding site]-[miR-221 binding site]-[ASO]-[smOPT]-[3′ box];
[hl56i (core)]-[U7promoter]-[ASO]-[smOPT]-[3′ box]; or
[DLX2.0]-[U7promoter]-[ASO]-[smOPT]-[3′ box].

26. The artificial expression construct of claim 8, wherein the artificial expression construct comprises or encodes a set of features selected from:
[hl56i (core)]-[U7promoter]-[miR-128 binding site]-[miR-221 binding site]-[SEQ ID NO: 1]-[smOPT]-[3′ box];
[DLX2.0]-[U7promoter]-[miR-128 binding site]-[miR-221 binding site]-[SEQ ID NO: 1]-[smOPT]-[3′ box];
[hl56i (core)]-[U7promoter]-[SEQ ID NO: 1]-[smOPT]-[3′ box];
[DLX2.0]-[U7promoter]-[SEQ ID NO: 1]-[smOPT]-[3′ box];
[hl56i (core)]-[U7promoter]-[miR-128 binding site]-[miR-221 binding site]-[SEQ ID NO: 2]-[smOPT]-[3′ box];
[DLX2.0]-[U7promoter]-[miR-128 binding site]-[miR-221 binding site]-[SEQ ID NO: 2]-[smOPT]-[3′ box];
[hl56i (core)]-[U7promoter]-[SEQ ID NO: 2]-[smOPT]-[3′ box];
[DLX2.0]-[U7promoter]-[SEQ ID NO: 2]-[smOPT]-[3′ box];
[hl56i (core)]-[U7promoter]-[miR-128 binding site]-[miR-221 binding site]-[SEQ ID NO: 3]-[smOPT]-[3′ box];
[DLX2.0]-[U7promoter]-[miR-128 binding site]-[miR-221 binding site]-[SEQ ID NO: 3]-[smOPT]-[3′ box];
[hl56i (core)]-[U7promoter]-[SEQ ID NO: 3]-[smOPT]-[3′ box];
[DLX2.0]-[U7promoter]-[SEQ ID NO: 3]-[smOPT]-[3′ box];
[hl56i (core)]-[U7promoter]-[miR-128 binding site]-[miR-221 binding site]-[SEQ ID NO: 4]-[smOPT]-[3′ box];
[DLX2.0]-[U7promoter]-[miR-128 binding site]-[miR-221 binding site]-[SEQ ID NO: 4]-[smOPT]-[3′ box];
[hl56i (core)]-[U7promoter]-[SEQ ID NO: 4]-[smOPT]-[3′ box];
[DLX2.0]-[U7promoter]-[SEQ ID NO: 4]-[smOPT]-[3′ box];
[hl56i (core)]-[U7promoter]-[miR-128 binding site]-[miR-221 binding site]-[SEQ ID NO: 5]-[smOPT]-[3′ box];
[DLX2.0]-[U7promoter]-[miR-128 binding site]-[miR-221 binding site]-[SEQ ID NO: 5]-[smOPT]-[3′ box];
[hl56i (core)]-[U7promoter]-[SEQ ID NO: 5]-[smOPT]-[3′ box];
[DLX2.0]-[U7promoter]-[SEQ ID NO: 5]-[smOPT]-[3′ box];
[hl56i (core)]-[U7promoter]-[miR-128 binding site]-[miR-221 binding site]-[SEQ ID NO: 6]-[smOPT]-[3′ box];
[DLX2.0]-[U7promoter]-[miR-128 binding site]-[miR-221 binding site]-[SEQ ID NO: 6]-[smOPT]-[3′ box];
[hl56i (core)]-[U7promoter]-[SEQ ID NO: 6]-[smOPT]-[3′ box];
[DLX2.0]-[U7promoter]-[SEQ ID NO: 6]-[smOPT]-[3′ box];
[hl56i (core)]-[U7promoter]-[miR-128 binding site]-[miR-221 binding site]-[SEQ ID NO: 7]-[smOPT]-[3′ box];
[DLX2.0]-[U7promoter]-[miR-128 binding site]-[miR-221 binding site]-[SEQ ID NO: 7]-[smOPT]-[3′ box];
[hl56i (core)]-[U7promoter]-[SEQ ID NO: 7]-[smOPT]-[3′ box];
[DLX2.0]-[U7promoter]-[SEQ ID NO: 7]-[smOPT]-[3′ box];
[hl56i (core)]-[U7promoter]-[miR-128 binding site]-[miR-221 binding site]-[SEQ ID NO: 8]-[smOPT]-[3′ box];
[DLX2.0]-[U7promoter]-[miR-128 binding site]-[miR-221 binding site]-[SEQ ID NO: 8]-[smOPT]-[3′ box];
[hl56i (core)]-[U7promoter]-[SEQ ID NO: 8]-[smOPT]-[3′ box];
[DLX2.0]-[U7promoter]-[SEQ ID NO: 8]-[smOPT]-[3′ box];
[hl56i (core)]-[U7promoter]-[miR-128 binding site]-[miR-221 binding site]-[SEQ ID NO: 9]-[smOPT]-[3′ box];
[DLX2.0]-[U7promoter]-[miR-128 binding site]-[miR-221 binding site]-[SEQ ID NO: 9]-[smOPT]-[3′ box];
[hl56i (core)]-[U7promoter]-[SEQ ID NO: 9]-[smOPT]-[3′ box];
[DLX2.0]-[U7promoter]-[SEQ ID NO: 9]-[smOPT]-[3′ box];
[h156i (core)]-[U7promoter]-[miR-128 binding site]-[miR-221 binding site]-[SEQ ID NO: 10]-[smOPT]-[3′ box];
[DLX2.0]-[U7promoter]-[miR-128 binding site]-[miR-221 binding site]-[SEQ ID NO: 10]-[smOPT]-[3′ box];
[hl56i (core)]-[U7promoter]-[SEQ ID NO: 10]-[smOPT]-[3′ box];
[DLX2.0]-[U7promoter]-[SEQ ID NO: 10]-[smOPT]-[3′ box];
[hl56i (core)]-[U7promoter]-[miR-128 binding site]-[miR-221 binding site]-[SEQ ID NO: 11]-[smOPT]-[3′ box];
[DLX2.0]-[U7promoter]-[miR-128 binding site]-[miR-221 binding site]-[SEQ ID NO: 11]-[smOPT]-[3′ box];
[hl56i (core)]-[U7promoter]-[SEQ ID NO: 11]-[smOPT]-[3′ box];
[DLX2.0]-[U7promoter]-[SEQ ID NO: 11]-[smOPT]-[3′ box];
[hl56i (core)]-[U7promoter]-[miR-128 binding site]-[miR-221 binding site]-[SEQ ID NO: 12]-[smOPT]-[3′ box];
[DLX2.0]-[U7promoter]-[miR-128 binding site]-[miR-221 binding site]-[SEQ ID NO: 12]-[smOPT]-[3′ box];
[hl56i (core)]-[U7promoter]-[SEQ ID NO: 12]-[smOPT]-[3′ box];
[DLX2.0]-[U7promoter]-[SEQ ID NO: 12]-[smOPT]-[3′ box];
[hl56i (core)]-[U7promoter]-[miR-128 binding site]-[miR-221 binding site]-[SEQ ID NO: 30]-[smOPT]-[3′ box];
[DLX2.0]-[U7promoter]-[miR-128 binding site]-[miR-221 binding site]-[SEQ ID NO: 30]-[smOPT]-[3′ box]. [hl56i (core)]-[U7promoter]-[SEQ ID NO: 30]-[smOPT]-[3′ box]; or
[DLX2.0]-[U7promoter]-[SEQ ID NO: 30]-[smOPT]-[3′ box].

27. The artificial expression construct of claim 8, wherein the artificial expression construct comprises:
the sequence as set forth in SEQ ID NO: 35 or a sequence having at least 95% sequence identity to the sequence as set forth in SEQ ID NO: 35;
the sequence as set forth in SEQ ID NO: 36 or a sequence having at least 95% sequence identity to the sequence as set forth in SEQ ID NO: 36; or
the sequence as set forth in SEQ ID NO: 37 or a sequence having at least 95% sequence identity to the sequence as set forth in SEQ ID NO: 37.

28. The artificial expression construct of claim 8, wherein the artificial expression construct is associated with a capsid that crosses a blood brain barrier.

29. The artificial expression construct of claim 28, wherein the capsid comprises PHP.eB, AAV9, AAVrh. 10, AAV-BR1, AAV-PHP.S, AAV-PHP.B, or AAV-PPS.

30. A vector comprising an artificial expression construct of claim 8.

31. The vector of claim 30, wherein the vector comprises a viral vector.

32. The vector of claim 31, wherein the viral vector comprises a recombinant adeno-associated viral (AAV) vector.

33. An adeno-associated viral (AAV) vector comprising at least one artificial expression construct of claim 8.

34. A transgenic cell comprising an antisense oligonucleotide (ASO) of claim 3, an artificial expression construct of claim 8, and/or a vector of claim 30.

35. The transgenic cell of claim 34, wherein the transgenic cell is a GABAergic neuron.

36. The transgenic cell of claim 35, wherein the GABAergic neuron is a pan-GABAergic neuron, a forebrain GABAergic neuron, a hippocampal GABAergic neuron, or a cortical GABAergic neuron.

37. The transgenic cell of claim 34, wherein the transgenic cell is murine, human, or non-human primate.

38. A non-human transgenic animal comprising an antisense oligonucleotide (ASO) of claim 3, an artificial expression construct of claim 8, a vector of claim 30, and/or a transgenic cell of claim 34.

39. The non-human transgenic animal of claim 38, wherein the non-human transgenic animal is a mouse or a non-human primate.

40. An administrable composition comprising a pharmaceutically acceptable carrier and an antisense oligonucleotide (ASO) of claim 3, an artificial expression construct of claim 8, a vector of claim 30, and/or a transgenic cell of claim 34.

41. A kit comprising an antisense oligonucleotide (ASO) of claim 3, an artificial expression construct of claim 8, a vector of claim 30, a transgenic cell of claim 34, and/or an administrable composition of claim 40.

42. A method for providing an antisense oligonucleotide (ASO) within a targeted population of cells in vivo or in vitro, the method comprising administering the administrable composition of claim 40 in a sufficient dosage and for a sufficient time to a sample or subject comprising the targeted population of cells thereby providing the ASO within the population of cells.

43. The method of claim 42, wherein the ASO is 12-24 nucleotides in length.

44. The method of claim 42, wherein the ASO is 15-21 nucleotides in length.

45. The method of claim 42, wherein the ASO is 18 nucleotides in length.

46. The method of claim 42, wherein the ASO is encoded by at least one of SEQ ID NOS: 1-12 or 30 or a sequence having at least 98% sequence identity to the sequence as set forth in SEQ ID NOs: 1-12 or 30.

47. The method of claim 42, wherein the administering comprises pipetting.

48. The method of claim 47, wherein the pipetting is to a brain slice.

49. The method of claim 48, wherein the brain slice comprises a GABAergic neuron.

50. The method of claim 49, wherein the GABAergic neuron comprises a pan-GABAergic neuron, a forebrain GABAergic neuron, a hippocampal GABAergic neuron, or a cortical GABAergic neuron.

51. The method of claim 48, wherein the brain slice is murine, human, or non-human primate.

52. The method of claim 42, wherein the administering comprises administering to a living subject.

53. The method of claim 52, wherein the living subject is a human, non-human primate, or a mouse.

54. The method of claim 52, wherein the living subject has an SCN1A-related disorder.

55. The method of claim 54, wherein the SCN1A-related disorder comprises Dravet Syndrome (DS); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); sick sinus syndrome 1; early infantile SCN1A encephalopathy; early infantile epileptic encephalopathy (EIEE); autism; hemiplegic migraine; arthrogryposis multiplex congenita (AMC); myoclonic-atonic epilepsy (MAE); epilepsy of infancy with migrating focal seizures (EIMFS); Rett syndrome; nonsyndromic epileptic encephalopathy (NEE); or intractable childhood epilepsy with generalized tonic-clonic seizures (ICE-GTC).

56. The method of claim 52, wherein the living subject is a pediatric patient.

57. The method of claim 52, wherein the living subject is less than 4 years old.

58. The method of claim 52, wherein the living subject is an adult.

59. The method of claim 52, wherein the living subject is a transgenic Scn1a^{R613X} mutant mouse.

60. The method of claim 42, wherein the administering to a living subject is through injection.

61. The method of claim 60, wherein the injection comprises intracerebroventricular (ICV) injection, intravenous injection, intraparenchymal injection into brain tissue, intra-cisterna magna (ICM) injection, or intrathecal injection.

62. The method of claim 42, wherein the composition is administered by intracerebroventricular (ICV) injection.

63. The method of claim 42, wherein the providing rescues voltage-gated sodium channel function in a cell in need thereof, with a proviso that the ASO does not comprise a sequence encoded by SEQ ID NO: 4.
